(19) United States
(12) Patent Application Publication
(10) Pub. No.: US 2024/0307036 A1
(43) Pub. Date: Sep. 19, 2024

(54) ULTRASONIC DIAGNOSTIC DEVICE, METHOD FOR OPERATING ULTRASONIC DIAGNOSTIC DEVICE, AND PROGRAM

(71) Applicant: GE Precision Healthcare LLC, Waukesha, WI (US)

(72) Inventors: Yukifumi Kobayashi, Hino (JP); Hiroshi Hashimoto, Hino (JP)

(21) Appl. No.: 18/605,178

(22) Filed: Mar. 14, 2024

(30) Foreign Application Priority Data
Mar. 15, 2023 (JP) 2023-040594

(51) Int. Cl. A61B 8/00; G06T 7/00

(52) U.S. Cl. CPC A61B 8/469; A61B 8/463; A61B 8/465; A61B 8/468; G06T 7/0012; G06T 2200/24; G06T 2207/10024; G06T 2207/10132; G06T 2207/20104; G06T 2207/30096

(57) ABSTRACT

An ultrasonic diagnostic device configured to (i) accept an input from a user selecting one image group from among the plurality of image groups, (ii) responsive to the input selecting an image group, display the ultrasound images included in the selected image group on the display, (iii) accept, through the user interface, an input for assigning a region of interest to an ultrasound image displayed on the display, (iv) responsive to the input assigning the region of interest, create a data set representing a temporal change in a feature in the region of interest based on the ultrasonic images included in the selected image group, and (v) arrange and integrate a plurality of data sets obtained by repeatedly executing operations (i) to (iv) in a time series, then create a data series representing a temporal change in a feature in the region of interest in the plurality of data sets.

### CROSS-REFERENCE TO RELATED APPLICATION

The present application claims priority to Japanese Patent Application No. 2023-040594, filed on Mar. 15, 2023. The entire contents of the above-listed application are incorporated by reference herein in their entirety.

### TECHNICAL FIELD

The present invention relates to an ultrasonic diagnostic device that creates a data set representing a temporal change in a feature in a region of interest, an operating method for the ultrasonic diagnostic device, and a program for the ultrasonic diagnostic device.

### BACKGROUND

An ultrasound examination may be performed using contrast-enhanced ultrasound, in which a contrast agent is injected into a subject to create a contrast image of the subject. In contrast-enhanced ultrasound, a region of interest (ROI) may be assigned to a target (for example, a tumor) in a contrast image and a time intensity curve may be generated for the region of interest. The time-intensity curve is also referred as a TIC or time-luminance curve. The time-intensity curve can be used for the differential diagnosis of tumors, etc.

In contrast-enhanced ultrasound, new diagnostic information can be obtained by conducting observations over a relatively long time period, for example, 5 minutes or 10 minutes after administration of a contrast agent. For example, it is said that the complexity of the vascular structure of a tumor, vascular resistance, etc. can be estimated by observing a gradual decrease in signal intensity from 2 minutes to 5 minutes after the administration of a contrast agent. In addition, hepatic function may potentially be quantified by comparing the accumulated contrast agent in the hepatic parenchyma immediately after administration, 5 minutes after administration, and 10 minutes after administration.

In order to obtain such diagnostic information, it is necessary to collect data over a relatively long period of time after the administration of the contrast agent. However, a longer examination time places a greater burden on the examiner and the subject.

When acquiring intermittent data, a change in signal intensity from the contrast agent is to be observed over a period of 10 minutes, ultrasonic scanning and image recording are not performed continuously for 10 minutes, but rather the first minute is captured continuously, followed by, for example, 5 second lengths of video every time a predetermined amount of time (for example, 1 minute) has elapsed. Therefore, since scanning is continuously performed for the first minute, it is necessary to acquire an image group including a large number of ultrasonic images. However, once the first minute has elapsed, an image group corresponding to a short time length of time, for example, approximately 5 seconds, may be acquired every time a predetermined time (for example, 1 minute) elapses. Therefore, once the images for the first minute have been acquired, it is only necessary to concentrate on image acquisition for a short period of time, reducing the burden on the examiner and the subject. After acquiring these image groups, the examiner creates a time-intensity curve for each image group, arranges the created time-intensity curves in a time series, and integrates them to display a single time-intensity curve. In this way, the examiner can obtain various types of diagnostic information by referring to a single time-intensity curve obtained by integration.

When creating a single integrated time-intensity curve, the user first selects an image group from the plurality of image groups obtained during the examination. Then, a region of interest is assigned to the selected image group and the position, size, and/or shape of the region of interest is adjusted while checking the data set in the assigned region of interest in order to determine the region of interest. By determining a region of interest for the selected image group in this way, a data set corresponding to the selected image group can be obtained. When the data set is obtained, the user selects the next image group from among the plurality of image groups. Then, a region of interest is determined for the selected next image group and a data set is obtained. Similarly, image groups are selected from the plurality of image groups in order to obtain data sets for all image groups. Then, a single integrated time-intensity curve can be created based on the data set obtained for each image group. In this case, if a desired data set can be obtained for all the image groups, a single integrated time-intensity curve is considered suitable for obtaining information useful for diagnosis. However, when the desired data set cannot be obtained for one or more image groups among the plurality of image groups obtained during an examination, a single integrated time-intensity curve may not be suitable for obtaining information useful for diagnosis. Therefore, in order to obtain a desired data set for all image groups, the user carefully examines the optimum position for the region of interest in a selected image group and selects the next image group once it has been determined that the desired data set has been obtained for the selected image group. However, it is not always easy for the user to determine whether a desired data set has been obtained for the selected image group. Therefore, when a user is performing the work of obtaining a data set for a certain image group, the user may wish to correct the data set for image groups on which the work of obtaining a data set has already been completed. In this case, if a single integrated time-intensity curve is created without performing the necessary correction work on the data sets, useful information for diagnosis may potentially not be obtained. One possible way of addressing this problem would be for the user to carefully perform the work of determining the optimum region of interest for each image group with sufficient time so that the work of correcting the data set does not have to be performed later. However, this is problematic in that such work increases the burden on the user, in addition to actually being difficult for a user to determine the region of interest so that the user does not need to correct the data sets later.

Therefore, there is a demand for the development of an ultrasonic diagnostic device that allows a user to easily perform correction work on data sets

### SUMMARY

According to an aspect, an ultrasonic diagnostic device may include: a user interface; a processor; a storage device configured to store a plurality of image groups, each image group including a plurality of ultrasound images arranged in a time series; and a display, wherein the processor is configured to: (i) accept, through the user interface, an input from a user selecting one image group from among the plurality of image groups, (ii) responsive to the input selecting an image group, display the ultrasound images included in the selected image group on the display, (iii) accept, through the user interface, an input for assigning a region of interest to an ultrasound image displayed on the display, (iv) responsive to the input assigning the region of interest, create a data set representing a temporal change in a feature in the region of interest based on the ultrasonic images included in the selected image group, and (v) arrange and integrate a plurality of data sets obtained by repeatedly executing operations (i) to (iv) in a time series, then create a data series representing a temporal change in a feature in the region of interest in the plurality of data sets, wherein the user input to select one image group includes a first input from the user to select a first image group of the plurality of image groups and a second input from the user to select a second image group of the plurality of image groups after accepting the first input, and wherein the user interface is configured to accept user input in order to reselect the first image group after the second input has been accepted, so that the user can perform correction work on the data set representing the temporal change in the feature in the first image group selected by the first input that was accepted before the second input.

According to another aspect, A method for operating an ultrasonic diagnostic device including a user interface, a processor, a storage device that stores a plurality of image groups, each image group including a plurality of ultrasound images arranged in a time series, and a display, the method may include (i) obtaining an input from a user selecting one image group from among the plurality of image groups; (ii) responsive to the user interface has accepted input from the user selecting the one image group, displaying the ultrasound images included in the selected image group on the display; (iii) receiving an input from a user performing an operation of assigning a region of interest to the ultrasound image displayed on the display; (iv) responsive to the user interface accepting input from the user assigning the region of interest, creating a data set representing a temporal change in a feature in the region of interest based on the ultrasonic images included in the selected image group, and (v) integrating a plurality of data sets obtained by repeatedly executing (i) to (iv) in time series, then creating a data series representing a temporal change in the feature in the region of interest in the plurality of data sets, the user input to select one image group including a first input from the user to select a first image group from the plurality of image groups and a second input from the user to select a second image group from the plurality of image groups after accepting the first input, and the method may further include: accepting, after the user interface has accepted the second input, input from the user to reselect the first image group so that the user can perform correction work on the data set representing the temporal change of the feature in the first image group selected in the first input before the second input was accepted.

According to yet another aspect, A non-transitory computer readable medium may be configured to be executed by a computer to: (i) obtain an input from a user through a user interface selecting one image group from among the plurality of image groups; (ii) responsive to the user interface accepting input from the user selecting the one image group, display the ultrasound images included in the selected image group on the display; (iii) receiving an input from a user performing an operation of assigning a region of interest to the ultrasound image displayed on the display; (iv) responsive to the user interface accepting input from the user assigning the region of interest, create a data set representing a temporal change in a feature in the region of interest based on the ultrasonic images included in the selected image group, and (v) integrate a plurality of data sets obtained by repeatedly executing (i) to (iv) in time series, then create a data series representing a temporal change in the feature in the region of interest in the plurality of data sets, the user input to select one image group including a first input from the user to select a first image group from the plurality of image groups and a second input from the user to select a second image group from the plurality of image groups after accepting the first input, and accepting, after the user interface has accepted the second input, input from the user to reselect the first image group so that the user can perform correction work on the data set representing the temporal change of the feature in the first image group selected in the first input before the second input was accepted.

### BRIEF DESCRIPTION OF THE DRAWINGS

FIG. 1 is a diagram illustrating a subject being scanned using an ultrasonic diagnostic device 1 according to an embodiment.

FIG. 2 is a block diagram of the ultrasonic diagnostic device 1 according to an embodiment.

FIG. 3 is an explanatory diagram of a time line on which a contrast image is acquired according to an embodiment.

FIG. 4 is an explanatory diagram of data acquired according to the time line illustrated in FIG. 3 according to an embodiment.

FIG. 5 is a diagram illustrating a flowchart for creating a time-intensity curve according to an embodiment.

FIG. 6 is an explanatory diagram of an example of an initial image according to an embodiment.

FIG. 7 is a diagram illustrating the screen after an image group 21 has been selected by a user according to an embodiment.

FIG. 8 is a diagram illustrating a parameter i assigned to an image group 21 according to an embodiment.

FIG. 9 is a diagram illustrating a user touching the TIC Analysis button 101 displayed on the touch panel 28 according to an embodiment.

FIG. 10 is a schematic diagram of an analysis screen displayed on the monitor 18 according to an embodiment.

FIG. 11 is an explanatory diagram of a region of interest according to an embodiment.

FIG. 12 is a diagram illustrating time-intensity curve 62 according to an embodiment.

FIG. 13 is a diagram illustrating time-intensity curve 63 according to an embodiment.

FIG. 14 is a diagram illustrating the user adjusting the scale and sizes of regions of interest 41 and 42 according to an embodiment.

FIG. 15 is a diagram illustrating an example of a time-intensity curve after the degree of smoothing has been adjusted according to an embodiment.

FIG. 16 is a diagram illustrating a situation in which the user has moved regions of interest 41 and 42 according to an embodiment.

FIG. 17 is an explanatory diagram of analysis information stored in memory according to an embodiment.

FIG. 18 is a diagram illustrating regions of interest 51 and 52 assigned to tissue to be compared with a target according to an embodiment.

FIG. 19 is an explanatory diagram of a method for selecting the next image group according to an embodiment.

FIG. 20 is a diagram illustrating a parameter i assigned to image group 22 according to an embodiment.

FIG. 21 is a diagram illustrating an analysis screen for analyzing a time-intensity curve of a selected image group 22 according to an embodiment.

FIG. 22 is an explanatory diagram of the analysis work performed by the user in step ST6 according to an embodiment.

FIG. 23 is an explanatory diagram of analysis information stored in memory according to an embodiment.

FIG. 24 is an explanatory diagram of a method for reselecting a previously selected image group 21 according to an embodiment.

FIG. 25 is a diagram illustrating a sub-menu 103 according to an embodiment.

FIG. 26 is a diagram illustrating an example of a correction screen displayed on the monitor 18 according to an embodiment according to an embodiment.

FIG. 27 is a diagram illustrating the position of the modified region of interest according to an embodiment.

FIG. 28 is an explanatory diagram of a method for selecting the next image group.

FIG. 29 is a diagram illustrating a parameter i assigned to image group 23 according to an embodiment.

FIG. 30 is a diagram illustrating an analysis screen for analyzing the time-intensity curve for selected image group 23 according to an embodiment.

FIG. 31 illustrates a sub-menu 103 with three selection buttons 104, 105, and 106 displayed according to an embodiment.

FIG. 32 is a diagram illustrating a sub-menu 103 on which J selection buttons are displayed according to an embodiment.

FIG. 33 is a diagram illustrating the user touching the Merge Graphs button 107 displayed on the touch panel 28 according to an embodiment.

FIG. 34 is a diagram illustrating a plurality of data sets displayed as a single integrated data series according to an embodiment.

FIG. 35 is a diagram in which lines 81, 82, 83, and 84 connecting time-intensity curves have been added according to an embodiment.

### DETAILED DESCRIPTION

Embodiments for carrying out the invention will be described below; however, the present invention is not limited to the following embodiment.

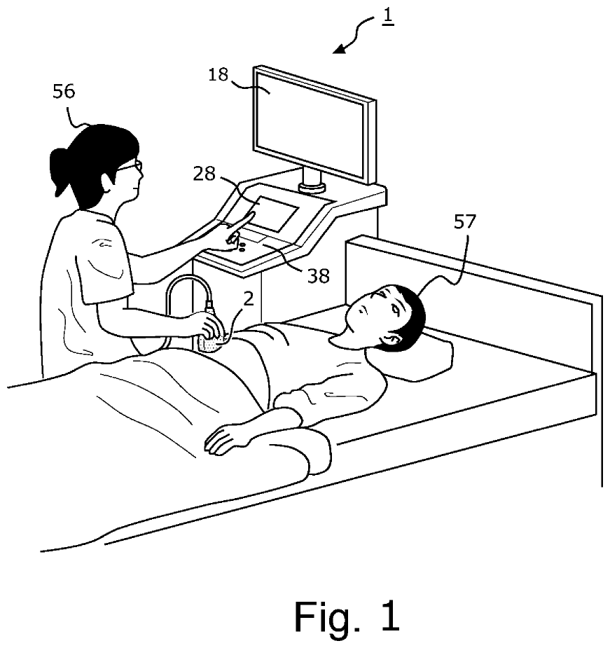

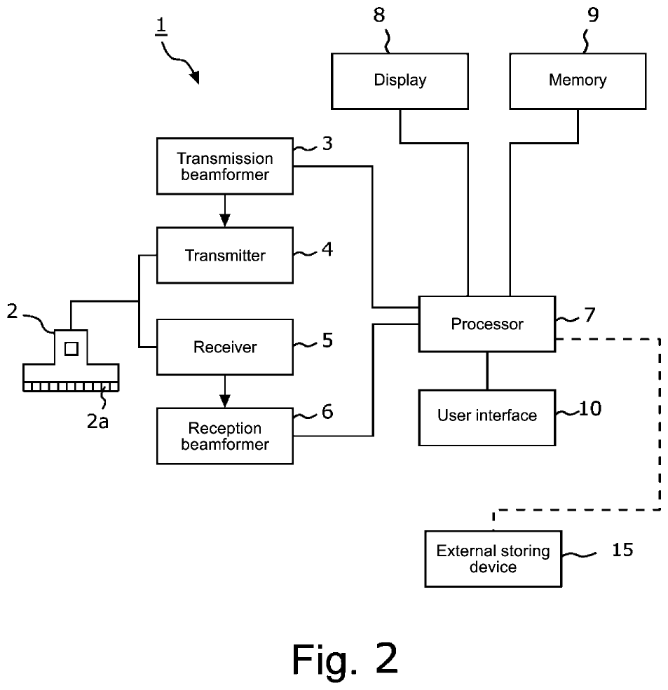

FIG. 1 is a diagram illustrating a subject being scanned using an ultrasonic diagnostic device 1 according to an embodiment of the present invention, while FIG. 2 is a block diagram of the ultrasonic diagnostic device 1.

The ultrasonic diagnostic device 1 has an ultrasonic probe 2, a transmission beamformer 3, a transmitter 4, a receiver 5, a reception beamformer 6, a processor 7, a display 8, a memory 9, and a user interface 10.

The ultrasonic probe 2 has a plurality of vibrating elements 2a arranged in an array. The transmission beamformer 3 and the transmitter 4 drive the plurality of vibrating elements 2a, which are arrayed within the ultrasonic probe 2, and ultrasonic waves are transmitted from the vibrating elements 2a. The ultrasonic waves transmitted from the vibrating elements 2a are reflected inside the subject 57 (see FIG. 1) and a reflection echo is received by the vibrating elements 2a. The vibrating elements 2a convert the received echo to an electrical signal and output this electrical signal as an echo signal to the receiver 5. The receiver 5 executes a prescribed process on the echo signal and outputs the echo signal to the reception beamformer 6. The reception beamformer 6 executes reception beamforming on the signal received through the receiver 5 and outputs echo data.

The reception beamformer 6 may be a hardware beamformer or a software beamformer. If the reception beamformer 6 is a software beamformer, the reception beamformer 6 may include one or more processors, including one or more: i) a graphics processing unit (GPU); ii) a microprocessor; iii) a central processing unit (CPU); iv) a digital signal processor (DSP); or v) another type of processor capable of executing logical operations. A processor configuring the reception beamformer 6 may be configured by a processor different from the processor 7 or may be configured by the processor 7.

The ultrasonic probe 2 may include an electrical circuit for performing all or a portion of transmission beamforming and/or reception beamforming. For example, all or a portion of the transmission beamformer 3, the transmitter 4, the receiver 5, and the reception beamformer 6 may be provided in the ultrasonic probe 2.

The processor 7 controls the transmission beamformer 3, the transmitter 4, the receiver 5, and the reception beamformer 6. Furthermore, the processor 7 is in electronic communication with the ultrasonic probe 2. The processor 7 controls which of the vibrating elements 2a is active and the shape of ultrasonic beams transmitted from the ultrasonic probe 2. The processor 7 is in electronic communication with the display 8. The processor 7 can process echo data to generate an ultrasonic image. The term “electronic communication” may be defined to include both wired and wireless communications. The processor 7 may include a central processing unit (CPU) according to one embodiment. According to another embodiment, the processor 7 may include another electronic component that may perform a processing function such as a digital signal processor, a field programmable gate array (FPGA), a graphics processing unit (GPU), another type of processor, and the like. According to another embodiment, the processor 7 may include a plurality of electronic components capable of executing a processing function. For example, the processor 7 may include two or more electronic components selected from a list of electronic components including a central processing unit, a digital signal processor, a field programmable gate array, and a graphics processing unit.

The processor 7 may also include a complex demodulator (not illustrated in the drawings) that demodulates RF data. In an another embodiment, demodulation may be executed in an earlier step in the processing chain.

Moreover, the processor 7 may generate various ultrasonic images (for example, a B-mode image, color Doppler image, M-mode image, color M-mode image, spectral Doppler image, elastography image, TVI image, strain image, and strain rate image) based on data obtained by processing via the reception beamformer 6. In addition, one or more modules can generate these ultrasonic images.

An image beam and/or an image frame may be saved and timing information may be recorded indicating when the data is retrieved to the memory. The module may include, for example, a scan conversion module that performs a scan conversion operation to convert an image frame from a coordinate beam space to display space coordinates. A video processor module may also be provided for reading an image frame from the memory while a procedure is being implemented on the subject and displaying the image frame in real-time. The video processor module may save the image frame in an image memory, while ultrasound images are read from the image memory and displayed on the display 8.

In the present Specification, the term “image” can broadly indicate both a visual image and data representing a visual image. Furthermore, the term “data” can include raw data, which is ultrasound data before a scan conversion operation, and image data, which is data after the scan conversion operation.

Note that the processing tasks described above handled by the processor 7 may be executed by a plurality of processors.

Furthermore, when the reception beamformer 6 is a software beamformer, a process executed by the beamformer may be executed by a single processor or may be executed by the plurality of processors.

The display 8 can be an LED (light emitting diode) display, an LCD (liquid crystal display), or an organic EL (electro-luminescence) display, etc. The display 8 displays an ultrasonic image. In the first embodiment, the display 8 includes a display monitor 18 and a touch panel 28, as illustrated in FIG. 1. However, the display 8 may be configured from a single display rather than a display monitor 18 and a touch panel 28. Moreover, two or more display devices may be provided in place of the display monitor 18 and the touch panel 28.

The memory 9 is any known data storage medium. In one example, the ultrasonic image display system includes a non-transitory storage medium and a transitory storage medium. In addition, the ultrasonic image display system may also include a plurality of memories. The non-transitory storage medium can be, for example, a non-volatile storage medium such as a hard disk drive (HDD) drive, a read only memory (ROM), etc. The non-transitory storage medium may include a portable storage medium such as a CD (compact disk) or a DVD (digital versatile disk). A program executed by the processor 7 is stored in the non-transitory storage medium. The transitory storage medium is a volatile storage medium such as random access memory (RAM).

The memory 9 stores one or more commands that can be executed by the processor 7. One or more commands cause the processor 7 to execute predetermined operations

Note that the processor 7 may also be configured so as to be able to connect to an external storing device 15 by a wired connection or a wireless connection. In this case, the command(s) causing execution by the processor 7 can be distributed to both the memory 9 and the external storing device 15 for storage.

The user interface 10 may accept input from an operator 56. For example, the user interface 10 accepts commands and information input from an operator 56. The user interface 10 includes a touch panel 28 and an operation panel 38. The operating panel 38 can be configured so as to include a keyboard, a hard key, a trackball, a rotary control, a soft key, etc. The touch panel can also display soft keys, buttons, etc.

The ultrasonic diagnostic device 1 is configured as described above.

Next, the operation of the ultrasonic diagnostic device 1 in the present example will be described. In the present embodiment, an example will be described in which the ultrasonic diagnostic device 1 operates so as to acquire data on contrast images in a subject and create a time-intensity curve based on the acquired contrast image data.

First, a method for acquiring data on contrast images will be described. Specifically, the contrast image data is acquired as follows.

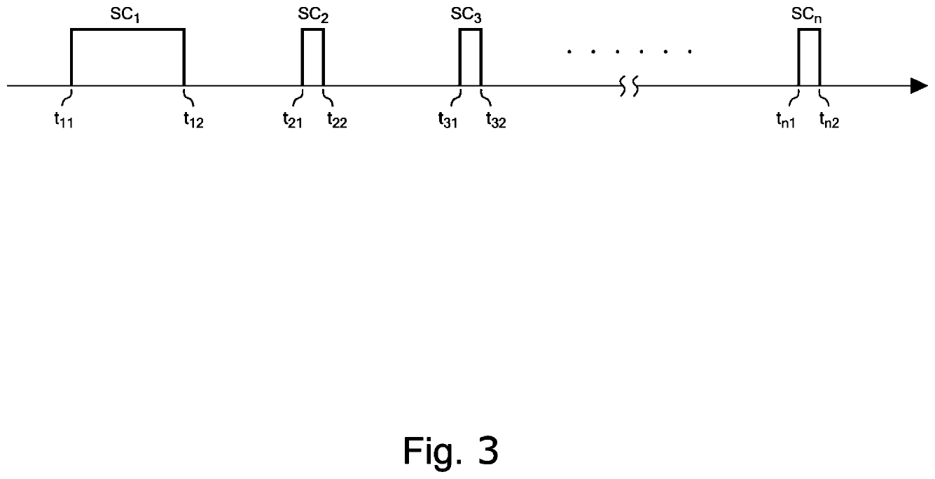

FIG. 3 is an explanatory diagram of a time line on which a contrast image is acquired.

The operator 56 injects a contrast agent into the subject 57 and acquires a contrast image. A contrast agent containing micro-bubbles as an active ingredient can be used as the contrast agent. The operator 56 operates the probe 2 to start acquisition of a contrast image at the examination site from time point t₁₁ (a time point at which the contrast agent is injected or a time point close to the time point at which the contrast agent is injected).

The processor 7 controls the ultrasonic probe 2 to transmit ultrasonic waves to the subject 57. The ultrasonic probe 2 receives ultrasonic waves (an echo) reflected by the contrast agent. The processor 7 performs predetermined processing on the echo signals and creates data indicating the signal intensity of the ultrasonic waves reflected by the contrast agent. Data indicating the signal intensity of the ultrasonic waves reflected by the contrast agent can be obtained as contrast image data.

The operator 56 acquires contrast image data intermittently over a predetermined time period from time point t₁₁. For example, the operator 56 acquires contrast images intermittently over a period from time t₁₁ to time tₙ₂. The length of time from time point t₁₁ to time point tₙ₂ can be set to, for example, approximately 10 minutes.

During the period from time t₁₁ to time tₙ₂, the operator 56 does not continuously perform ultrasonic scanning and image recording, but rather performs data collection at the predetermined times. In the present embodiment, the acquisition SC₁ of data on the subject 57 is continuously performed from time point t₁₁ to time point t₁₂, after which the acquisition SC₂ to SCₙ of data is performed for several seconds every time a certain time period elapses. In the present embodiment, the subject 57 is scanned in accordance with the time line illustrated in FIG. 3.

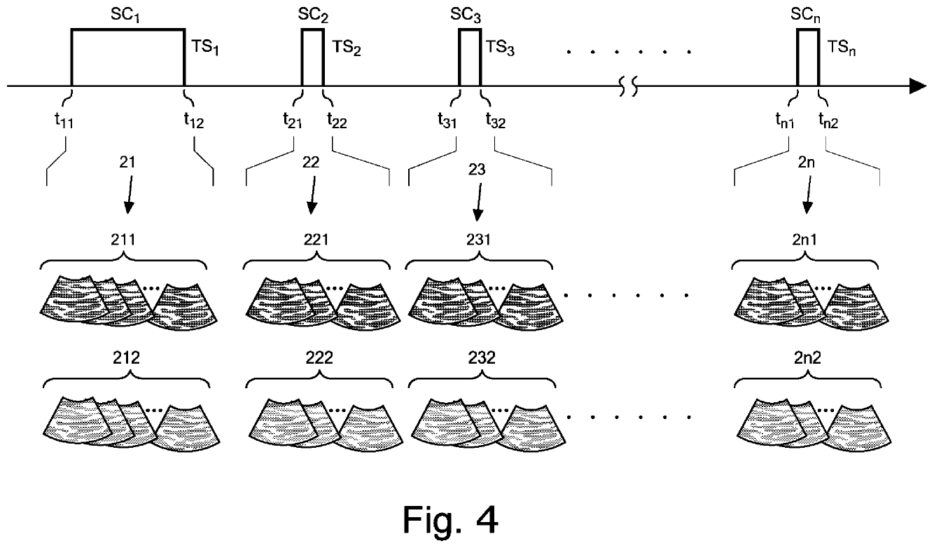

FIG. 4 is an explanatory diagram of data acquired according to the time line illustrated in FIG. 3.

First, the operator 56 administers a contrast agent, continuously performs scanning between time points t₁₁ and t₁₂, and acquires data of an image group 21 including a plurality of ultrasonic images. The time length between time points t₁₁ and t₁₂ is represented by “TS₁”, with the time length TS₁ being, for example, 60 seconds. Between time points t₁₁ and t₁₂, the processor 7 controls the ultrasonic probe 2 to transmit ultrasonic waves to the subject 57. The ultrasonic probe 2 receives ultrasonic waves (an echo) reflected by the contrast agent. The processor 7 performs predetermined processing on the echo signals and creates an ultrasonic image indicating the signal intensity of the ultrasonic waves reflected by the contrast agent. The ultrasonic image can be, for example, a cross-sectional image of the liver in the subject 57. The processor 7 stores the acquired data of the image group 21 in the memory 9. In the present embodiment, two types of ultrasonic images are created as ultrasound images. One is a contrast image in which nonlinear signals included in echo signals reflected by the contrast agent are enhanced, while the other is a B-mode image. Therefore, the image group 21 contains an image group 211 including a plurality of contrast images arranged in a time series and an image group 212 including a plurality of B-mode images arranged in a time series.

After acquiring the data from time points t₁₁ to t₁₂, the operator 56 interrupts the acquisition of data and stands by until time point t₂₁ at which the next data acquisition is started following injection of the contrast agent. Then, the operator 56 acquires data between time points t₂₁ and t₂₂ (time length TS₂). Time length TS₂ can be, for example, 5 seconds. The processor 7 performs predetermined processing on the echo signals to create image group 22. Image group 22 contains an image group 221 including a plurality of contrast images arranged in a time series and an image group 222 including a plurality of B-mode images arranged in a time series. The data for image group 22 is stored in the memory 9.

After acquiring data from time points t₂₁ to t₂₂, the operator 56 interrupts the data acquisition and stands by until time point t₃₁ at which the next data acquisition is started following injection of the contrast agent. Then, the operator 56 acquires data between time points t₃₁ and t₃₂ (time length TS₃). Time length TS₃ can be, for example, 5 seconds. The processor 7 performs predetermined processing on the echo signals to create image group 23. Image group 23 contains an image group 231 including a plurality of contrast images arranged in a time series, along with an image group 232 including a plurality of B-mode images arranged in a time series. The data for image group 23 is stored in the memory 9.

Subsequently, the step of acquiring data after standing by for a certain period of time is repeatedly executed.

Finally, the operator 56 acquires information during a period from time tₙ₁ to time tₙ₂ (time length TSₙ). The processor 7 performs predetermined processing on the echo signals to create image group 2n. Image group 2n contains an image group 2n1 including a plurality of contrast images arranged in a time series and an image group 2n2 including a plurality of B-mode images arranged in a time series. The data for image group 2n is stored in the memory 9.

Examination data on the subject 57 is acquired in this manner.

Data obtained by scanning the subject 57 is stored in the memory 9.

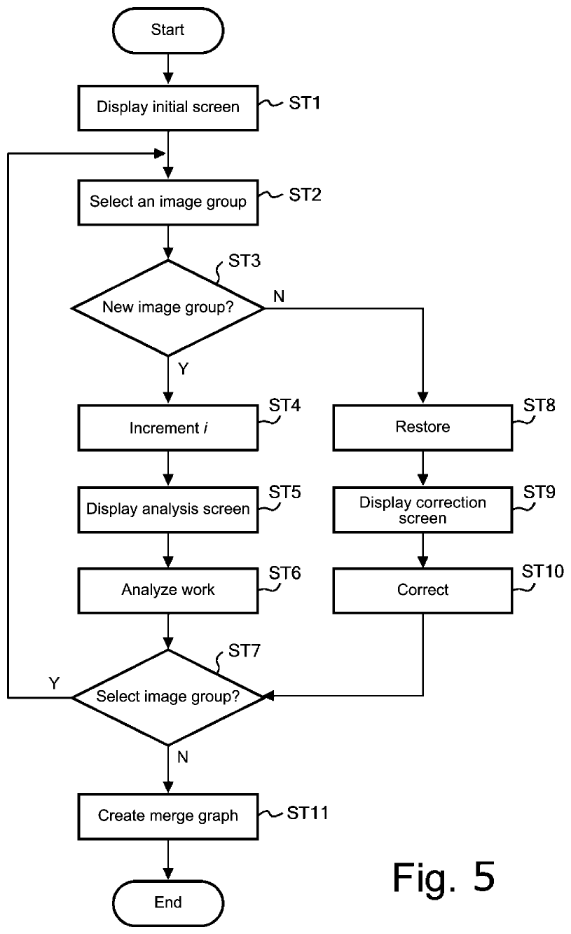

Next, the operator 56 creates a time-intensity curve based on the data acquired from the subject 57. This method of creating a time-intensity curve will now be described with reference to the flowchart in FIG. 5.

FIG. 5 is a diagram illustrating a flowchart for creating a time-intensity curve.

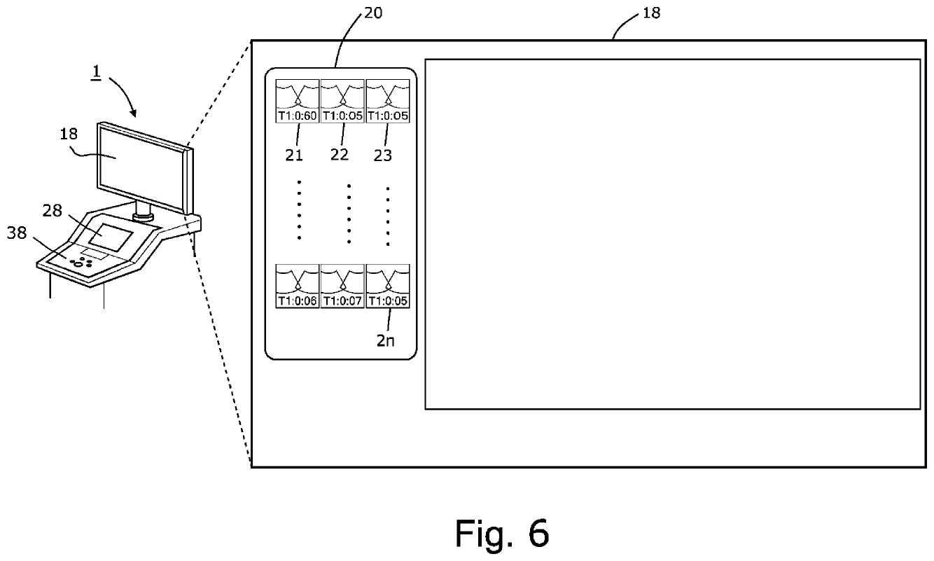

In step ST1, the user (for example, the operator 56, another operator, and/or a doctor) operates the user interface 10 to display an initial screen for creating a time-intensity curve on the monitor 18 (see FIG. 6).

FIG. 6 is an explanatory diagram illustrating an example of an initial screen. FIG. 6 illustrates an enlarged view of the initial screen displayed on the monitor 18.

A thumbnail region 20 is shown on the screen of the monitor 18. In the thumbnail region 20, thumbnails corresponding to n image groups from image group 21 to image group 2n acquired by scanning the subject 57 are displayed. In FIG. 6, among image groups 21 to 2n, four image groups 21, 22, 23, and 2n are illustrated as representatives using reference numerals. After displaying the initial screen, the process proceeds to step ST2.

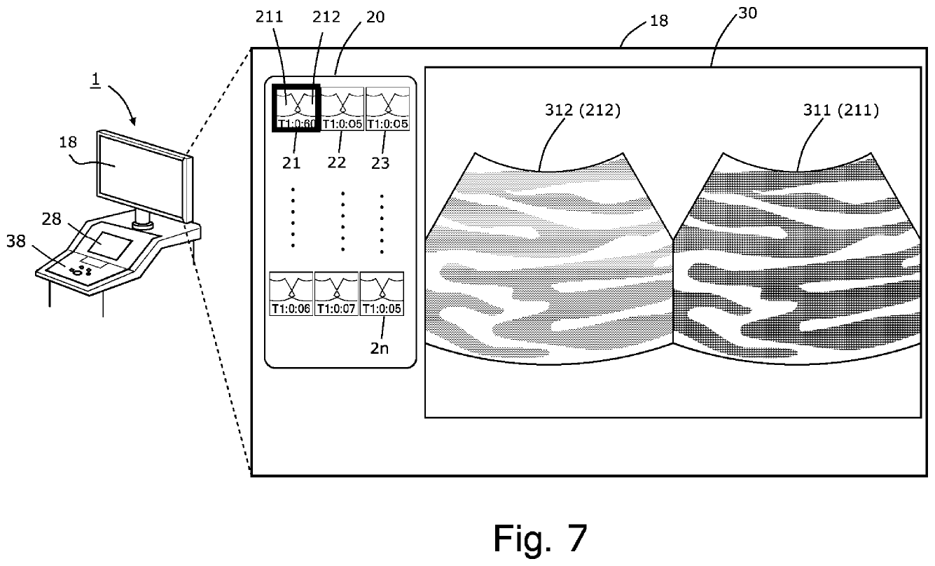

In Step ST2, the user operates the user interface 10 to input a command to select the image group to be used for creating a time-intensity curve from among the plurality of image groups 21 to 2n. The user can select an image group to be used for creating a time-intensity curve from among the plurality of image groups 21 to 2n by operating, for example, a trackball or various buttons on the operation panel 38. For example, when selecting image group 21, the user operates the operation panel 38 to input a command to select image group 21. When this command is input, the user interface 10 accepts input from the user to select image group 21 from among the plurality of image groups 21 to 2n. When the user interface 10 accepts input from the user, image group 21 is selected and the next screen is displayed on the monitor 18 (see FIG. 7).

FIG. 7 is a diagram illustrating the screen after image group 21 has been selected by the user.

An image display region 30 is illustrated to the right of the thumbnail region 20 displayed on the screen of the monitor 18. When the user selects image group 21, the processor reproduces an image from selected image group 21 in the image display region 30. Image group 21 contains an image group 211 of contrast images and an image group 212 of B-mode images, with a contrast image 311 from image group 211 displayed in the right half of the image display region 30 and a B-mode image 312 from the image group 212 displayed in the left half. Image groups 211 and 212 are reproduced as video having time length TS₁ (see FIG. 4). In this way, the user can confirm images acquired between time points t₁₁ and t₁₂ as video. In the image display region 30, one contrast image 311 (an image of one frame selected from a plurality of frames) in image group 211 of contrast images may be displayed as a still image, while one B-mode image 312 (an image of one frame selected from a plurality of frames) in image group 212 of B-mode images may be displayed as a still image. Alternatively, only one of the group of contrast images 211 and the group of B-mode images 212 may be displayed. Once an image group has been selected, the process proceeds to step ST3.

In step ST3, the processor determines whether the selected image group is an image that has been a previously selected or an image group that is being selected for the first time (that is, a newly selected). If the selected image group is an image group that has been previously selected, the process proceeds to step ST8, whereas if the selected image group is an image group that is being selected for the first time (that is, newly selected), the process proceeds to step ST4. Here, the image group 21 is not an image group that has been previously selected but rather an image group that is being selected for the first time (newly selected). Therefore, the process proceeds to step ST4.

In step ST4, the processor assigns an identifier to the image group selected by the user. Any identifier can be used as the identifier as long as it can identify a newly selected image group. In the present embodiment, an example is described in which an image number is used as an identifier.

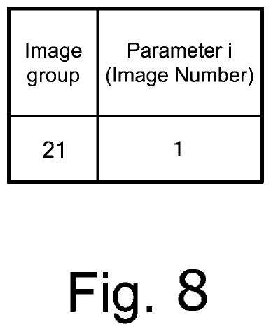

In step ST4, the processor increments parameter i representing the image number of the new selected image group. Here, it is assumed that parameter i is set to an initial value i=0. Consequently, the processor increments parameter i from the initial value i=0 to i=1. In this way, as illustrated in FIG. 8, the processor assigns parameter i=1 (that is, image number 1) to image group 21 and stores it in the memory. After parameter i=1 has been assigned to image group 21, the process proceeds to step ST5.

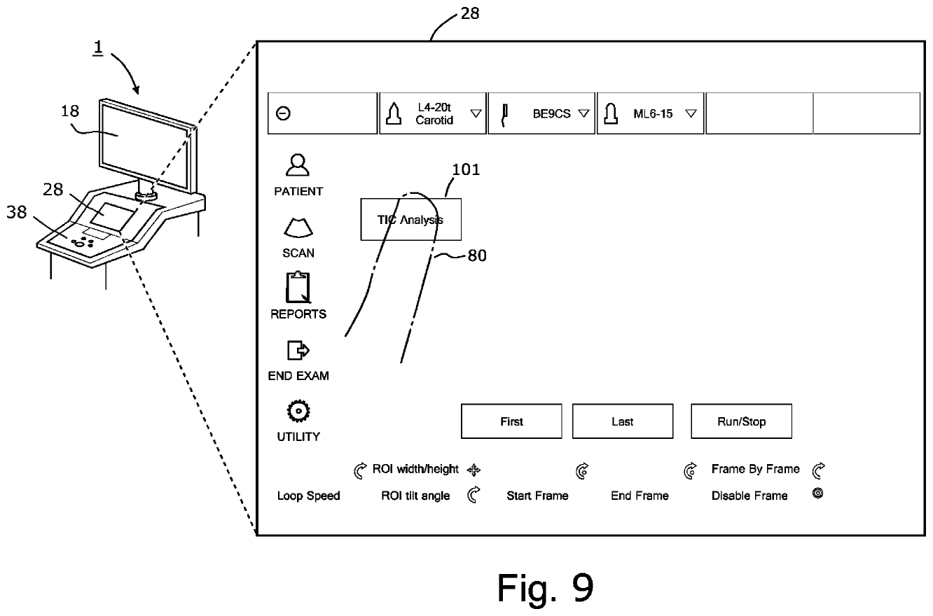

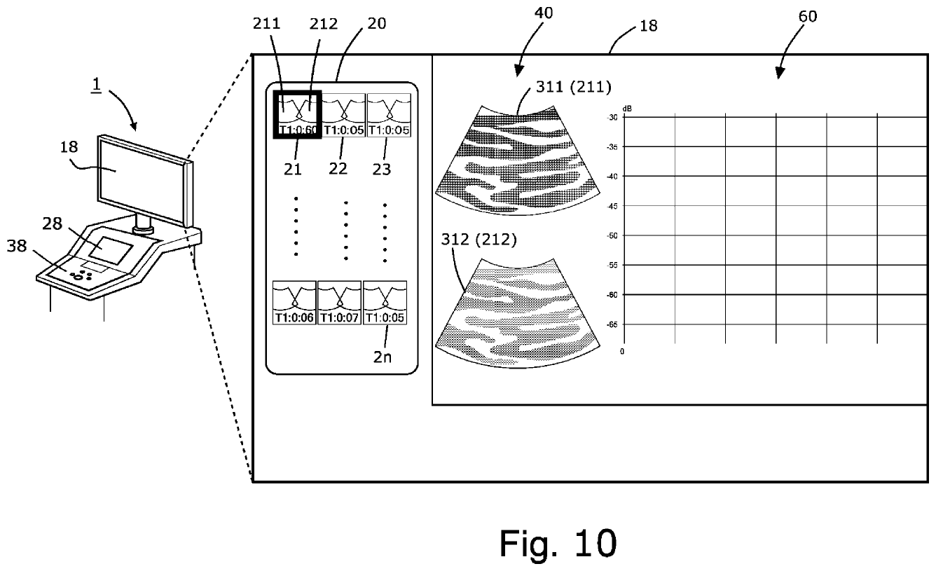

In step ST5, the user operates the user interface to input a command to display an analysis screen for analyzing the time-intensity curve of selected image group 21 on the monitor 18. This command can be input, for example, by operating the touch panel 28. Specifically, as illustrated in FIG. 9, the user can input a command by touching the TIC Analysis button 101 displayed on the touch panel 28 with a finger 80. When the user interface receives input from the user (a touch of the button 101 by the user), the processor displays an analysis screen for analyzing the time-intensity curve of image group 21 on the monitor 18. FIG. 10 is a schematic diagram of the analysis screen displayed on the monitor 18.

A thumbnail region 20, an image display region 40, and a TIC region 60 are displayed on the monitor 18. In the thumbnail region 20, image groups 21 to 2n are displayed. In the image display region 40, image group 211 (contrast image 311) and image group 212 (B-mode image 312) included in selected image group 21 are displayed. The TIC region 60 is a region in which a time-intensity curve is displayed. After the analysis screen has been displayed on the monitor 18, the process proceeds to step ST6.

In step ST6, the user performs analysis work. The specific details of this analysis work are described below.

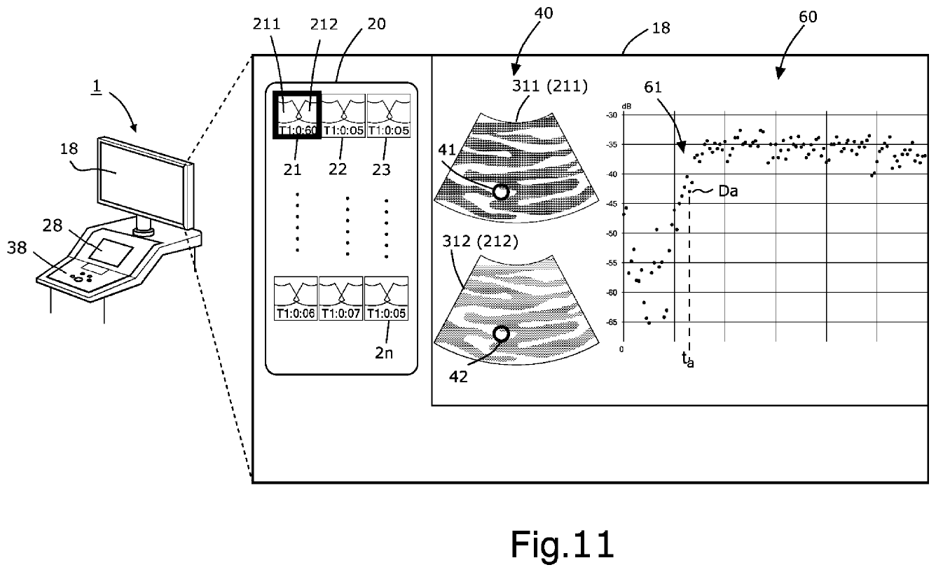

First, the user assigns a region of interest to the contrast image 311 (and the B-mode image 312) displayed on the monitor 18. FIG. 11 is an explanatory diagram of a method for assigning a region of interest.

While referring to the contrast image 311 (and the B-mode image 312), the user confirms the position of a target (for example, a tumor or a lesion) in the examination region. Then, the user operates the user interface to input a command for assigning a region of interest to a location of a target of the contrast image 311 (and the B-mode image 312). When the user interface receives input from the user to assign a region of interest, the processor 7 displays figures representing the regions of interest 41 and 42 on the contrast image 311 (and the B-mode image 312). In FIG. 11, the regions of interest 41 and 42 are represented by circles. The position of the region of interest 42 in the B-mode image 312 corresponds to the position of the region of interest 41 in the contrast image 311. The user can simultaneously move the regions of interest 41 and 42 by manipulating the user interface.

Once a region of interest has been assigned, the processor 7 calculates a feature in the region of interest 41 with respect to each contrast image 311 (each frame) of the image group 211. The feature in the region of interest 41 can be calculated as, for example, a value representing luminance information in the region of interest 41. In one example, the feature in the region of interest 41 can be calculated as an average value, a median value, or a standard deviation of the pixel values in the region of interest 41. In this way, a feature in the region of interest 41 can be calculated for each contrast image 311 in image group 211.

Once the feature in the region of interest has been calculated, the processor displays the feature in the region of interest 41 calculated for each contrast image 311 in the image group 211 as illustrated in the TIC region 60 of FIG. 11. In the TIC region 60, a data set 61 representing the temporal change in the feature in the region of interest 41 calculated for image group 211 of contrast image 311 is shown. The horizontal axis of the data set 61 represents time, while the vertical axis represents the feature in the region of interest. In FIG. 11, each data point included in the data set 61 is indicated by a black circle. Each data point in the data set 61 represents a feature in the region of interest 41 in each contrast image 311 of image group 211. For example, data point Da represents the feature in the region of interest 41 calculated based on the contrast image at time point ta.

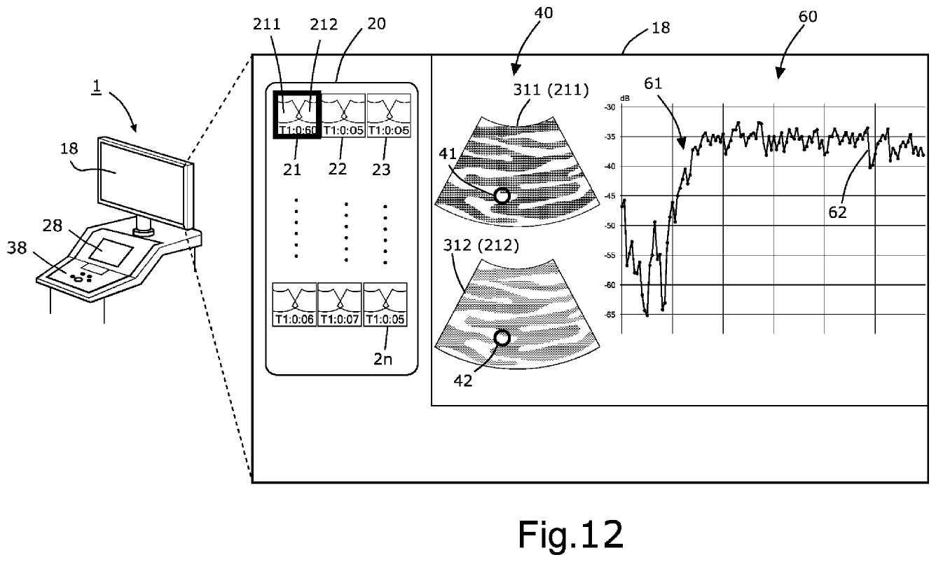

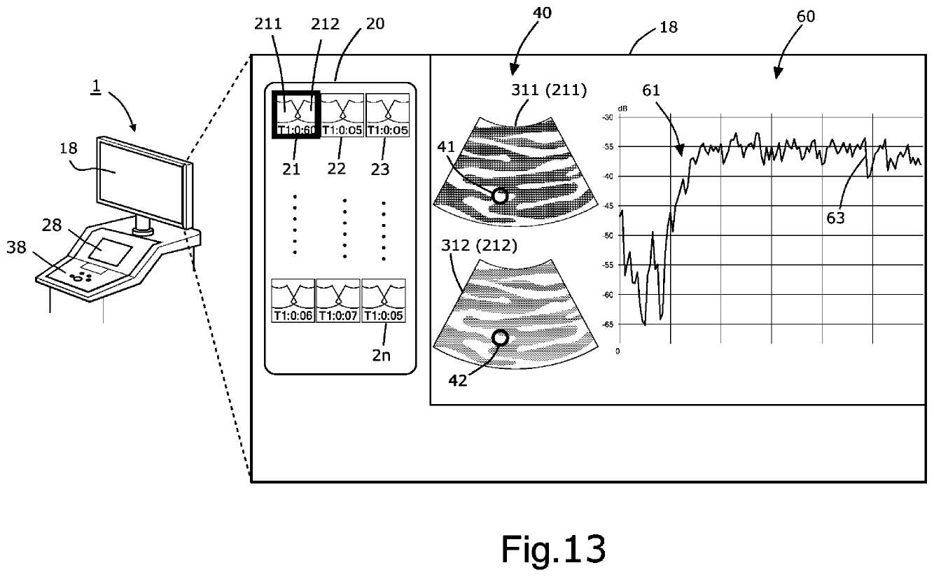

The user can visually recognize the temporal change in the feature in the region of interest 41 by referring to the data set 61 displayed in the TIC region 60. In FIG. 11, the temporal change in the feature in the region of interest 41 is indicated by dots arranged in a time series. However, in order to make it easy to visually recognize the temporal change in the feature in the region of interest 41, as illustrated in FIG. 12, a time-intensity curve 62 may be displayed in which points adjacent to each other in the time axis direction are connected by a line. In addition, as illustrated in FIG. 13, a time-intensity curve 63 may be displayed in which a data set representing a temporal change in the feature in the region of interest 41 is indicated only by a line without points. As described above, the temporal change in a feature in the region of interest 41 can be displayed in various ways. In the following description, for convenience of explanation, it is assumed that the temporal change in the feature in the region of interest is displayed using only a line as illustrated in FIG. 13.

As described above, by referring to the time-intensity curve 63 displayed in the TIC region 60, the user can visually recognize how the feature in the region of interest 41 has changed over time.

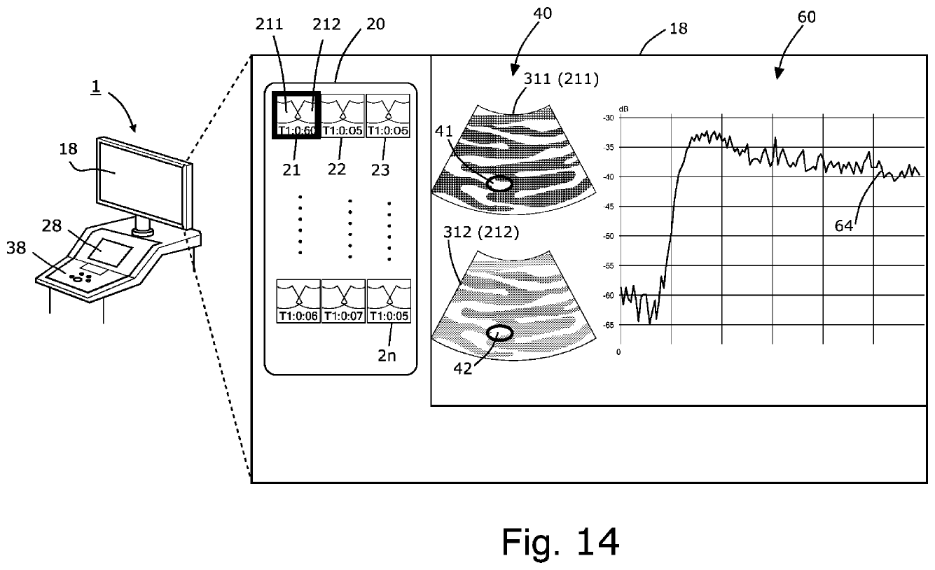

The user can change the conditions for creating the time-intensity curve as necessary. For example, the user can change the size and shape of the region of interest in accordance with the size of the target, the shape of the target, and the like. If the target has, for example, an elliptical shape, the user can adjust the size and shape of the region of interest so as to correspond to the elliptical shape of the target. FIG. 14 is a diagram illustrating the user adjusting the size of regions of interest 41 and 42. FIG. 14 illustrates an example in which the shapes of regions of interest 41 and 42 are adjusted so as to be elliptical. In this way, the user can adjust the size and shape of regions of interest 41 and 42 based on the size and shape of the target. Once the sizes and shapes of regions of interest 41 and 42 have been adjusted, the processor displays a time-intensity curve 64 corresponding to the adjusted region of interest 41 as illustrated in FIG. 14.

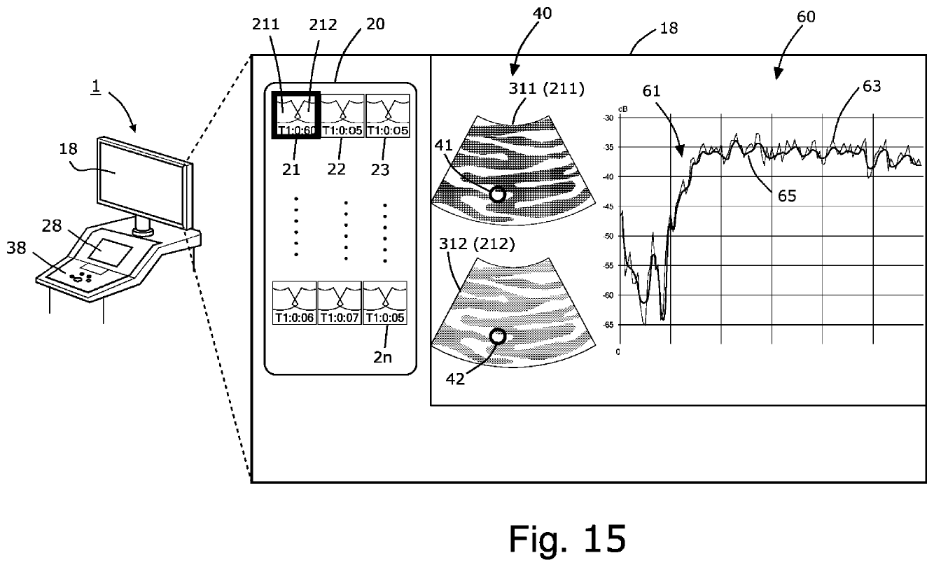

The user can also adjust the degree of smoothing for the time-intensity curve. For example, by referring to the time-intensity curve 63 (see FIG. 13) displayed on the monitor 18, the user can recognize how the feature in the regions of interest have changed over time, but may wish to adjust the degree of smoothing of the time-intensity curve 63 as necessary. For example, the user may want to make the waveform of the time-intensity curve 63 smoother or, conversely, may want to make the waveform of the time-intensity curve 63 rougher. In this case, the user can operate the user interface to input a command to execute a smoothing process for adjusting the degree of smoothing for the time-intensity curve. When the user interface receives input from the user to adjust the degree of smoothing for the time-intensity curve, the processor 7 executes smoothing processing so that the degree of smoothing for the time-intensity curve is adjusted, and displays the time-intensity curve. FIG. 15 is a diagram illustrating an example of a time-intensity curve after the degree of smoothing has been adjusted. In FIG. 15, the time-intensity curve after the degree of smoothing has been adjusted is indicated by reference number “65.” (In FIG. 15, for reference, the time-intensity curve 63 before the degree of smoothing has been adjusted is indicated by a thin line in order to clarify the effect of smoothing processing. The time-intensity curve 63 may not be displayed in the TIC region 60.)

By adjusting the degree of smoothing for the time-intensity curve, the user can visually recognize the trend in the temporal change of the feature in the regions of interest with case. The smoothing processing can use, for example, the moving average method for calculating the average value of m data points adjacent to each other in the time axis direction. By increasing or decreasing the value of m, the degree of smoothing for the time-intensity curve can be adjusted. In this way, the user can adjust the smoothness of the time-intensity curve to his or her own liking.

When examining, for example, a pulsating site in the subject or a region that moves due to the breathing motion of the subject, the user assigns a region of interest to coincide with a target (for example, a tumor or a lesion); however, the target moves due to the movement of the examination region, so the position of the target may be displaced from the position in the region of interest. If the positional deviation between the target and the region of interest becomes too large, it may potentially not be possible to obtain a time-intensity curve which properly reflects the temporal change in the feature in the target. Therefore, when a region of interest is assigned to a moving site such as the heart or the abdomen, motion compensation for the region of interest may be performed so that the region of interest can follow the motion of the target. When the user operates the user interface to input a command for performing motion compensation on the region of interest and the user interface receives input from the user to perform motion compensation on the region of interest, the processor executes motion compensation on the region of interest so that the region of interest moves with the motion of the target. Therefore, because the region of interest is positioned over the target even when the examination region moves, it is possible to obtain a time-intensity curve in which the temporal change in the feature of the target is correctly reflected.

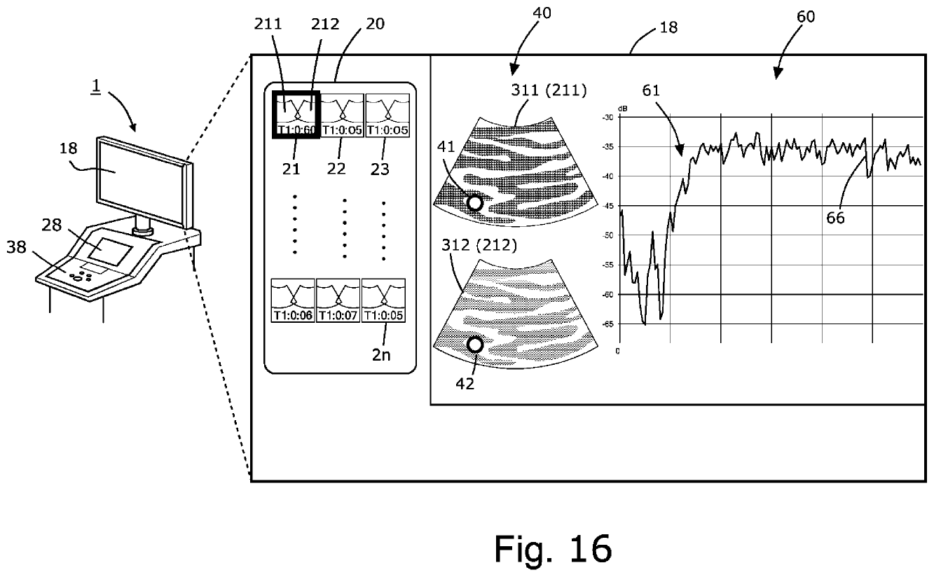

Should the user wish to adjust the position of the region of interest, the user can move the position of the region of interest to the desired position by operating the user interface. FIG. 16 is a diagram illustrating the regions of interest 41 and 42 moved by the user. FIG. 16 illustrates an example in which the user has moved the regions of interest 41 and 42 to the left with respect to their original positions (see FIG. 15). In this way, the user can freely move the region of interest to any position on the image. When the position of the region of interest 41 is moved, as illustrated in FIG. 16, the processor can display a time-intensity curve 66 representing the temporal change in the feature in the region of interest 41 after the movement.

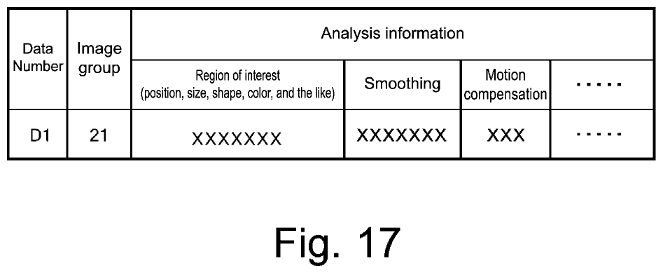

As described above, the user sets the region of interest on the image with reference to the location of the target, adjusting the position of the region of interest with reference to the time-intensity curve. In addition, the user adjusts the size and shape of the region of interest or adjusts the degree of smoothing for the time-intensity curve as necessary. In this way, the user analyzes image group 21. When analysis information (position, size, shape, and color of the region of interest, degree of smoothing, and whether the region of interest is motion-corrected or not, etc.) for image group 21 with respect to the time-intensity curve has been determined, the user stores the determined analysis information in the memory. For example, the user operates the user interface to input a command to store the analysis information in the memory. When the user interface receives input from the user to store the analysis information in the memory, the processor stores the analysis information in the memory. FIG. 17 is an explanatory diagram of the analysis information stored in a memory. Examples of analysis information stored in the memory include the position, size, shape, and color of the region of interest, the degree of smoothing, and whether motion compensation has been performed on the region of interest. However, the analysis information is not limited to these types of analysis information, with other analysis information capable of being stored in the memory instead of or in addition to these types of analysis information. The analysis information is stored in the data number D1 column. In addition, the processor maps the analysis information to the selected image group 21 and stores it in the memory.

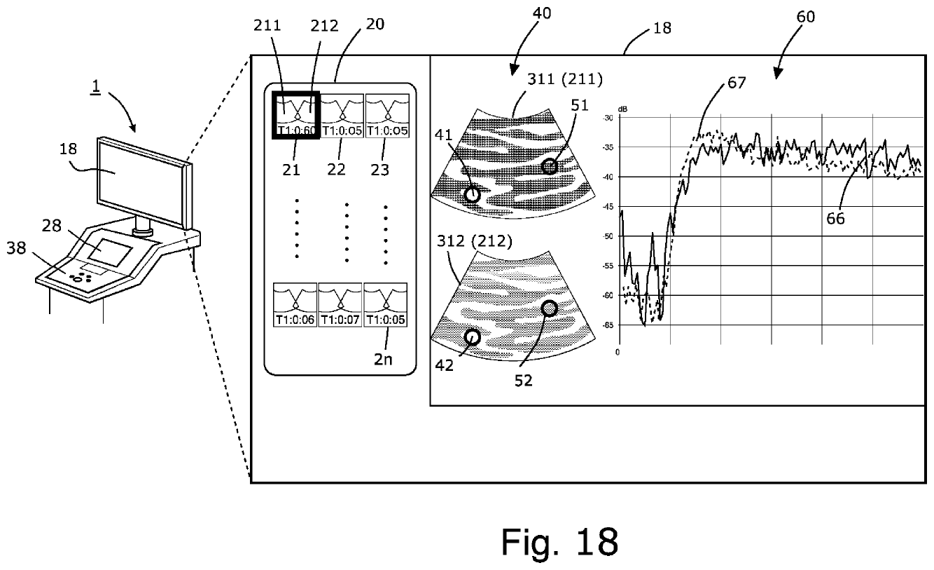

The user may also assign other regions of interest as necessary. For example, there may be a case in which the user wishes to create not only a time-intensity curve of a target (for example, a tumor or a lesion) but also a time-intensity curve of tissue to be compared with the target in order to diagnose an examination region. In such a case, the user may also assign other regions of interest. Here, a time-intensity curve is created for tissue to be compared with the target. Thus, the user assigns a region of interest to the target tissue to be compared. FIG. 18 is a diagram of regions of interest 51 and 52 that have been assigned to the target tissue to be compared. After regions of interest 51 and 52 have been assigned, the feature in the region of interest 51 is calculated and a time-intensity curve 67 representing the temporal change in the feature in region of interest 51 is displayed. In FIG. 18, the time-intensity curve 67 of the target tissue to be compared is indicated by a dashed line. When the user can determine analysis information for the image group 21 related to the time-intensity curve 67, the user stores the determined analysis information in the memory. The processor stores the analysis information in the memory so that the analysis information for image group 21 related to the time-intensity curve 67 (the position, size, shape, color, degree of smoothing, the presence or absence of motion compensation in the region of interest, etc.) is added to the data number D1 column (see FIG. 17) corresponding to image group 21. Therefore, analysis information when a region of interest has been assigned to the target and analysis information when a region of interest has been assigned to the tissue to be compared with the target are stored under data number D1.

When the user assigns another region of interest, the user assigns the region of interest according to the above-described method. In the event all necessary regions of interest have been assigned, the process proceeds to step ST7. Here, it is assumed that all necessary regions of interest have been assigned. Therefore, the process proceeds to step ST7.

In step ST7, the user determines whether to select another image group. If an image group is to be selected, the process returns to step ST2, while if an image group is not to be selected, the process proceeds to step ST11. Here, the user decides to select another image group. Therefore, the process returns to step ST2.

In step ST2, the user selects the next image for analyzing the time-intensity curve.

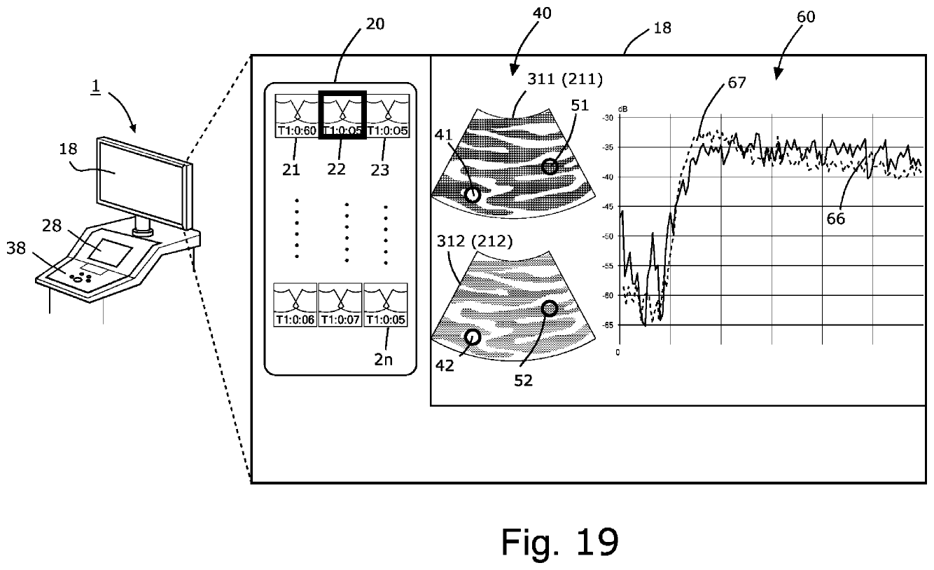

FIG. 19 is an explanatory diagram of a method for selecting the next image group. The user operates the user interface to input a command to select the next image group for analyzing the time-intensity curve from among image groups 21 to 2n. Here, the user selects image group 22 as the next image group. Thus, the user operates the user interface to input a command to select image group 22 from among image groups 21 to 2n. For example, the user can input a command to select image group 22 by operating a trackball or various buttons of the operation panel 38. When the user inputs a command, the user interface receives input from the user to select image group 22 from among the plurality of image groups 21 to 2n and image group 22 is selected. Once image group 22 has been selected, the process proceeds to step ST3.

In step ST3, the processor determines whether the selected image group 22 is an image group that has been previously selected or an image group that is being selected for the first time (that is, a newly selected). Image group 22 is not an image group that has been previously selected but rather an image group that is being selected for the first time (newly selected). Therefore, the process proceeds to step ST4.

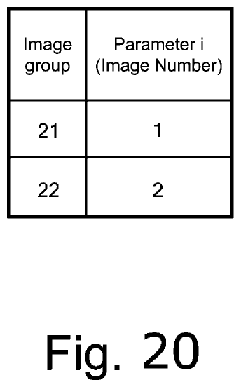

In step ST4, the processor increments parameter i representing the image number of the new selected image group. Since the current value of parameter i is i=1, the processor increments parameter i from i=1 to i=2. Then, as illustrated in FIG. 20, the processor assigns parameter i=2 to image group 22 and stores it in the memory. After parameter i=2 has been assigned to image group 22, the process proceeds to step ST5.

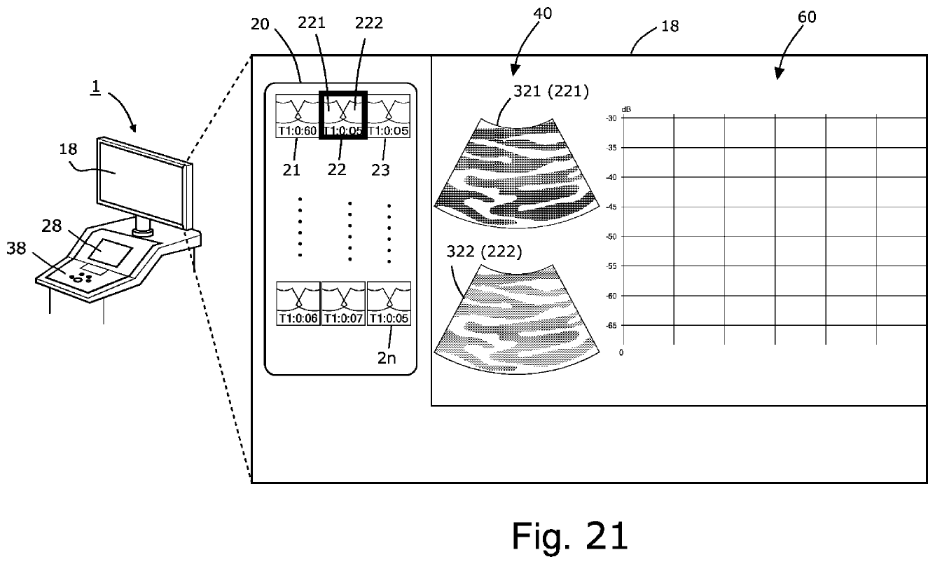

In step ST5, the processor 7 reads the selected image group 22 and displays an analysis screen for analyzing the time-intensity curve of the selected image group 22 on the monitor 18 (see FIG. 21).

FIG. 21 is a diagram illustrating an analysis screen for analyzing the time-intensity curve of the selected image group 22.

Image group 22 includes an image group 221 of contrast images and an image group 222 of B-mode images. Contrast image 321 in image group 221 is displayed in the upper part of the image display region 40, while B-mode image 322 in image group 222 is displayed in the lower part of the image display region 40. Image groups 221 and 222 are reproduced as video with time length TS₂ (see FIG. 4). In the image display region 40, a single contrast image 321 (an image of one frame selected from a plurality of frames) in image group 221 may be displayed as a still image, while a single B-mode image 322 in image group 222 may be displayed as a still image. Alternatively, only one image group 221 of contrast images and image group 222 of B-mode images may be displayed.

After displaying contrast image 321 and B-mode image 322 of image group 22, the process proceeds to step ST6.

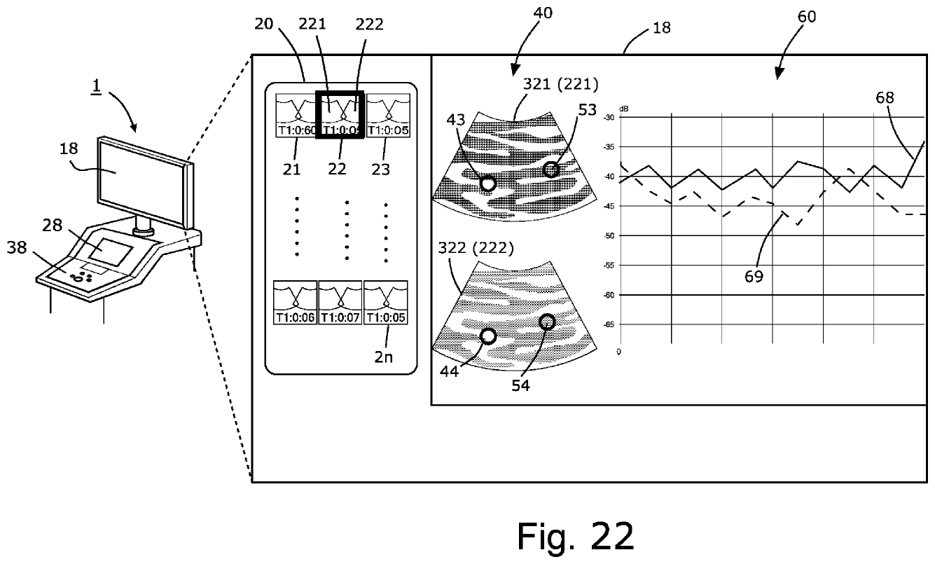

In step ST6, the user performs an analysis operation on the selected image group 22 (see FIG. 22).

FIG. 22 is an explanatory diagram of the analysis work performed by the user in step ST6.

While referring to the contrast image 321 (and B-mode image 322) displayed on the monitor 18, the user confirms the position of a target (for example, a tumor or a lesion) in the examination region. Then, the user operates the user interface to input a command for assigning a region of interest to a location of a target of the contrast image 321 (and B-mode image 322). For example, the user can assign a region of interest on a contrast image and B-mode image by operating a trackball or various buttons of the operation panel 38. When the user interface receives input from the user to assign a region of interest, the processor 7 displays a graphic figure representing regions of interest 43 and 44 on the contrast image 321 (and B-mode image 322). The position of the region of interest 44 in the B-mode image 322 corresponds to the position of the region of interest 43 in the contrast image 321. The user can simultaneously move the regions of interest 43 and 44 by manipulating the user interface.

The processor 7 calculates a feature in the region of interest 43 for each contrast image 321 (each frame) of the image group 221. The feature may be, for example, an average value, a median value, or a standard deviation of pixel values included in the region of interest 43. Then, as illustrated in the TIC region 60 of FIG. 22, the processor displays a data set (time-intensity curve 68) representing a temporal change in the feature in the region of interest 43.

The user adjusts the position of the region of interest with reference to the time-intensity curve, etc. In addition, the user adjusts the size and shape of the region of interest or adjusts the degree of smoothing for the time-intensity curve as necessary.

In this way, the user determines the analysis information for image group 22 with respect to the time-intensity curve 68.

The user also assigns regions of interest 53 and 54 to the target tissue to be compared and creates a time-intensity curve 69. Then, analysis information for image group 22 relating to the time-intensity curve 69 is also determined.

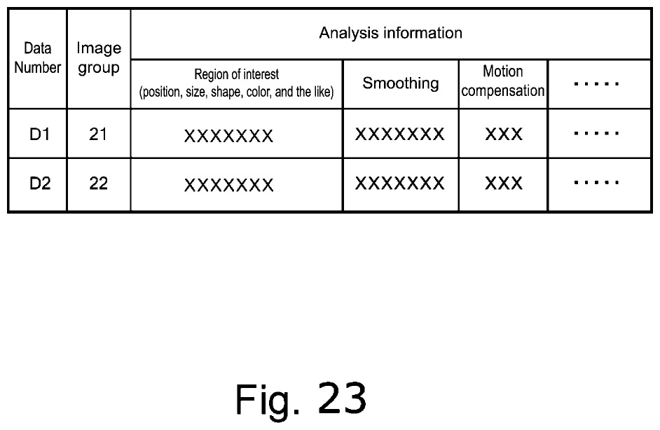

When the user determines the analysis information for image group 22 with respect to the time-intensity curves 68 and 69, the user operates the user interface to input a command to store the analysis information in the memory. When the user interface receives input from the user to store the analysis information in the memory, the processor stores the analysis information in the memory. FIG. 23 is an explanatory diagram of analysis information stored in a memory. The processor maps the analysis information for image group 22 related to time-intensity curve 68 and the analysis information of image group 22 related to time-intensity curve 69 to image group 22 and stores them under data number D2 column. Once the analysis information for image group 22 has been stored, the process proceeds to step ST7.

In step ST7, the user determines whether to select another image group. For example, when the user wants to select image group 23, the user can select the next image group 23 by operating the user interface.

During the work of analyzing the time-intensity curve for each image group, the user may determine that he or she wants to check analysis information for a previously selected image group (for example, image group 21) again and perform correction work on the data set of the previously selected image group (for example, the data set 61 for image group 21). For example, when making a determination from the position of region of interest 43 (see FIG. 22) in the currently selected image group 22 or from the waveform of the time-intensity curve 68, the user may feel that it is better to modify data group 61 (see, for example, FIG. 13) for previously selected image group 21.

However, referring to FIG. 22, the currently selected image group is image group 22, with contrast image 321 and B-mode image 322 from image group 22 displayed in the image display region 40 of the monitor 18 and the data set (time-intensity curves 68 and 69) for image group 22 displayed in the TIC region 60. Therefore, when image group 22 has been selected, the data set of previously selected image group 21 cannot be corrected.

Therefore, in the present embodiment, the user interface is configured so as to allow the user to perform correction work on the data set (time-intensity curve) of a previously selected image group (for example, image group 21) as necessary. Specifically, the user interface is configured so as to accept input from the user to reselect previously selected image group 21 even after accepting input from the user to select image group 22.

Here, the flow will be described for the case in which the user in step ST7 wants to reselect the previously selected image group 21.

In step ST7, if the user wants to reselect previously selected image group 21, the process returns to step ST2.

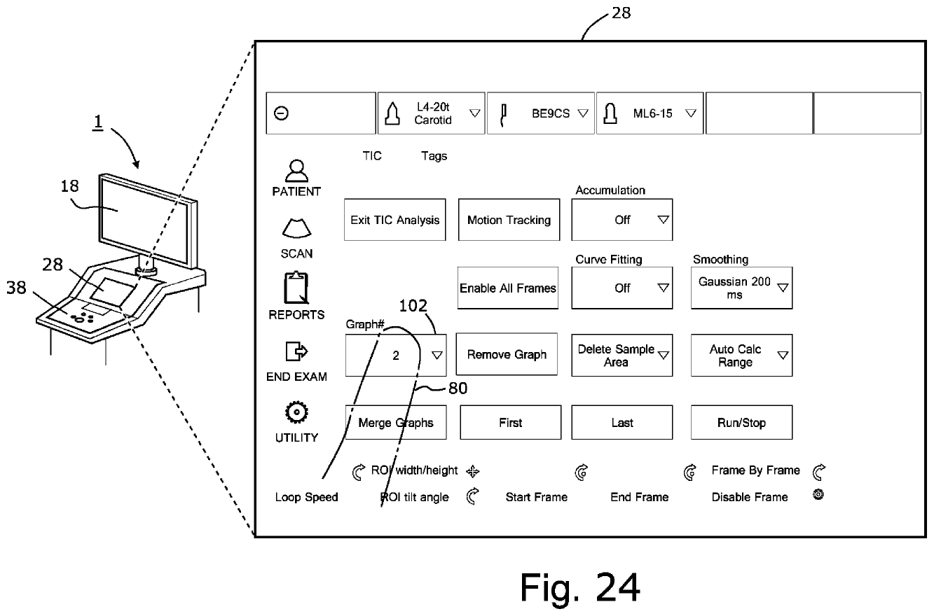

In Step ST2, the user operates the touch panel 28 to reselect previously selected image group 21 (see FIG. 24).

FIG. 24 is an explanatory diagram of a method for reselecting previously selected image group 21.

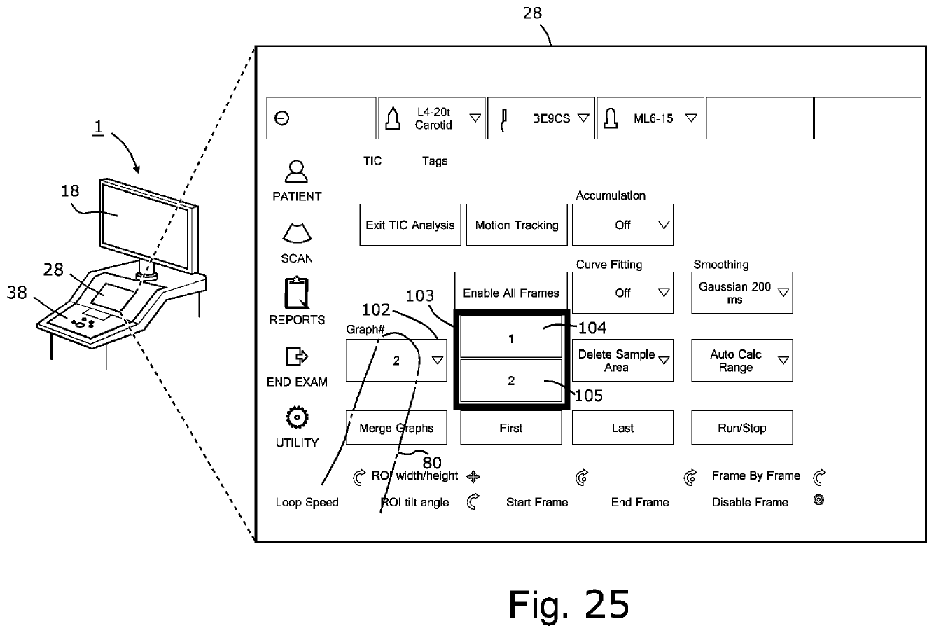

When the user wants to modify the data set for previously selected image group 21, the user operates the user interface to input a command to reselect previously selected image group 21. In the present embodiment, in order to select previously selected image group 21, the user first touches the button 102 on the touch panel 28 by finger 80 as illustrated in FIG. 24. When the user touches the button 102, as illustrated in FIG. 25, a sub-menu 103 is displayed allowing the user to reselect an image group previously selected by the user. Two selection buttons 104 and 105 are displayed in the sub-menu 103. Selection button 104 includes image number 1. Image number 1 indicates that selection button 104 is a button for allowing the user to reselect image group 21 (see FIG. 20) to which the image number 1 (parameter i=1) has been assigned. Selection button 105 includes image number 2. Image number 2 indicates that selection button 105 is a button for allowing the user to reselect image group 22 to which the image number 2 (parameter i=2) has been assigned. In this case, since the user intends to correct image group 21, the user touches selection button 104. When the user touches selection button 104, the touch panel 28 receives input from the user to reselect image group 21 corresponding to image number 1 and image group 21 is selected. Once image group 21 has been selected, the process proceeds to step ST3.

In step ST3, the processor determines whether selected image group 21 is an image group that has been previously selected or an image group that is being selected for the first time (that is, newly selected). Image group 21 is not a newly selected image but rather a previously selected image. Therefore, the process proceeds to step ST8.

In step ST8, the processor reads and restores the analysis information mapped to image group 21 (the analysis information in the data number D1 column illustrated in FIG. 23) from the memory. Once the analysis information has been restored, the process proceeds to step ST9.

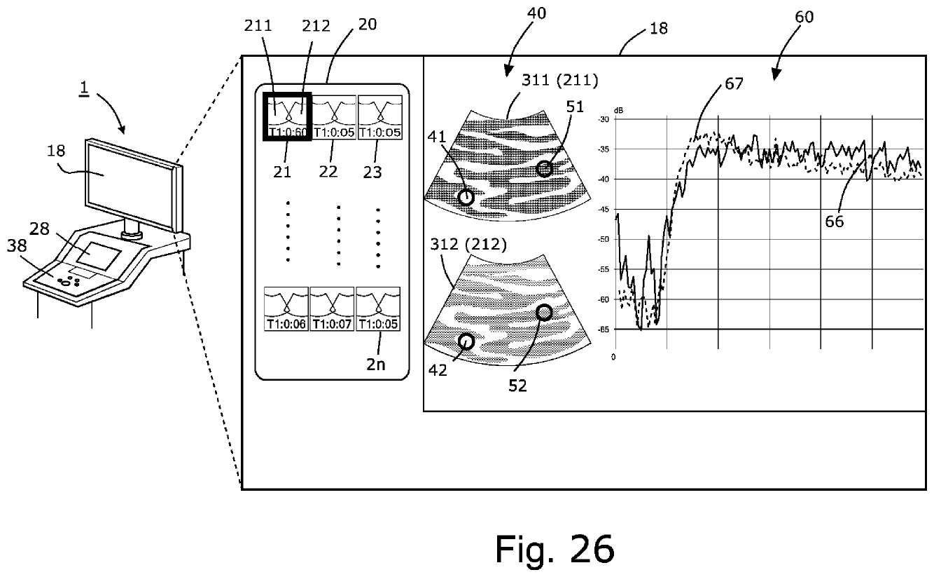

In step ST9, the processor displays a correction screen for image group 21 on the monitor 18 (see FIG. 26).

FIG. 26 is a diagram illustrating the correction screen displayed on the monitor 18. The processor displays the correction screen for image group 21 on the monitor 18 based on the analysis information for the restored image group 21.

Contrast image 311 and B-mode image 312 from image group 21 reselected are displayed in the image display region 40 of the monitor 18. Regions of interest 41 and 51 assigned when the user newly selected image group 21 are displayed in contrast image 311, with regions of interest 42 and 52 assigned when the user newly selected image group 21 are displayed in B-mode image 312. In the TIC region 60, the time-intensity curve 66 for region of interest 41 and the time-intensity curve 67 for region of interest 51 in the contrast image 311 are also displayed. After displaying the correction screen, processing proceeds to step ST10.

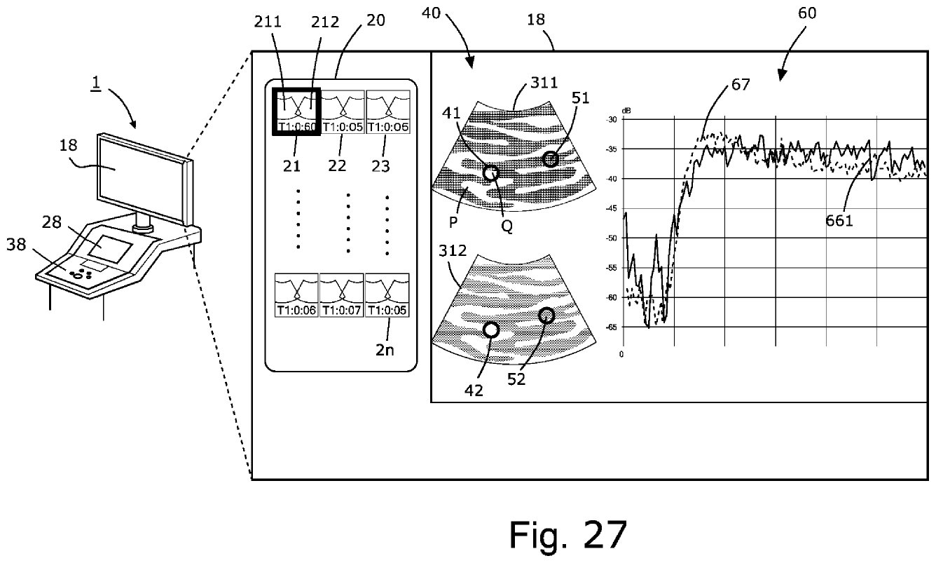

In step ST10, the user checks the position of region of interests and the waveform of the time-intensity curves displayed on the screen, then determines whether or not to perform correction work on image group 21 (the time-intensity curves 66 and 67). If the user decides to perform correction work, the user operates the user interface to input a correction command. For example, when the user determines that the current position of region of interest 41 deviates from the position in the target, the user operates the user interface to input a command for correcting the position of the region of interest. When the user interface receives input from the user to correct the position of the region of interest, the processor corrects the position of the region of interest based on user input (see FIG. 27).

FIG. 27 is a diagram illustrating the position of the corrected region of interest. The user can input a command to correct the position of region of interest 41 by operating a trackball or various buttons in the operation panel 38, for example. When the user interface receives input from the user to correct the position of region of interest 41, the processor moves region of interest 41 from position P to position Q based on user input. When the position of region of interest 41 has been moved, the processor calculates the luminance value in region of interest 41 based on pixel values in region of interest 41 after the position has been corrected and displays a data set (time-intensity curve 661) representing the temporal change in the feature in region of interest 41 at position Q.

In this way, the user can check the data set (time-intensity curve 661) of the region of interest after correction. The user confirms the data set (time-intensity curve) of region of interest 41 while further correcting the position of region of interest 41 as necessary. In this way, the user can correct the position of region of interest 41.

The user can also perform other correction work as necessary. Other correction work includes, for example, adding a new region of interest, changing the size, shape, or color of a region of interest, changing the degree of smoothing, and whether motion compensation has been performed on a region of interest.

In this way, the user can perform correction work on the data set representing the temporal change of the feature in a region of interest. After performing correction work, the user operates the user interface to input a command to update the analysis information stored in the memory to the corrected analysis information. When this command has been input, the processor stores the corrected analysis information in the memory, and as a result, the analysis information is updated. Once the correction work has been completed, the process returns to step ST7.

In step ST7, the user determines whether to select another image group. If an image group is to be selected, the process returns to step ST2, while if an image group is not to be selected, the process proceeds to step ST11. Here, the user decides to select another image group. Therefore, the process returns to step ST2.

In step ST2, the user selects the next image for analyzing the time-intensity curve.

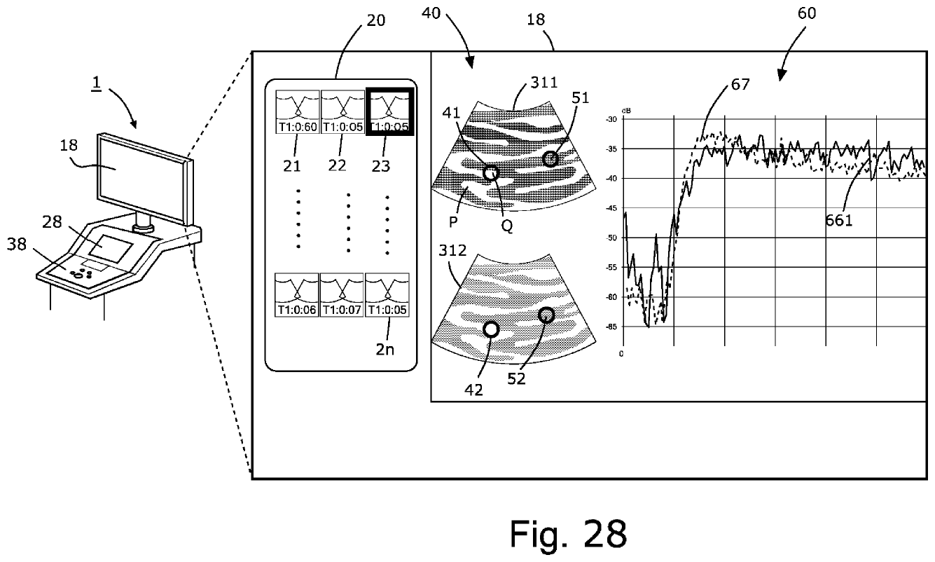

FIG. 28 is an explanatory diagram of a method for selecting the next image group. The user operates the user interface to input a command to select the next image group for analyzing the time-intensity curve from among image groups 21 to 2n. Here, the user selects image group 23 as the next image group. Thus, the user operates the user interface to input a command to select image group 23 from among image groups 21 to 2n. The user can input a command to select image group 23 by operating a trackball or various buttons on the operation panel 38, for example. When the user has input a command, the user interface receives input from the user to select image group 23 from among image groups 21 to 2n and image group 23 is selected. Once image group 22 has been selected, the process proceeds to step ST3.

In step ST3, the processor determines whether selected image group 23 is an image group that has been previously selected or an image group that is being selected for the first time (that is, newly selected). Image group 23 is not an image group that has been previously selected but a (newly selected) image group that is being selected for the first time. Therefore, the process proceeds to step ST4.

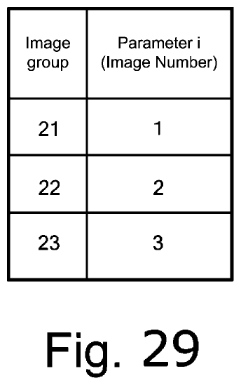

In step ST4, the processor increments the parameter i representing the image number for newly selected image group 23. Since the current value of parameter i is i=2, the processor increments parameter i from i=2 to i=3. Then, as illustrated in FIG. 29, the processor assigns parameter i=3 to image group 23 and stores parameter i=3 in the memory. Once parameter i=3 has been assigned to image group 23, the process proceeds to step ST5.

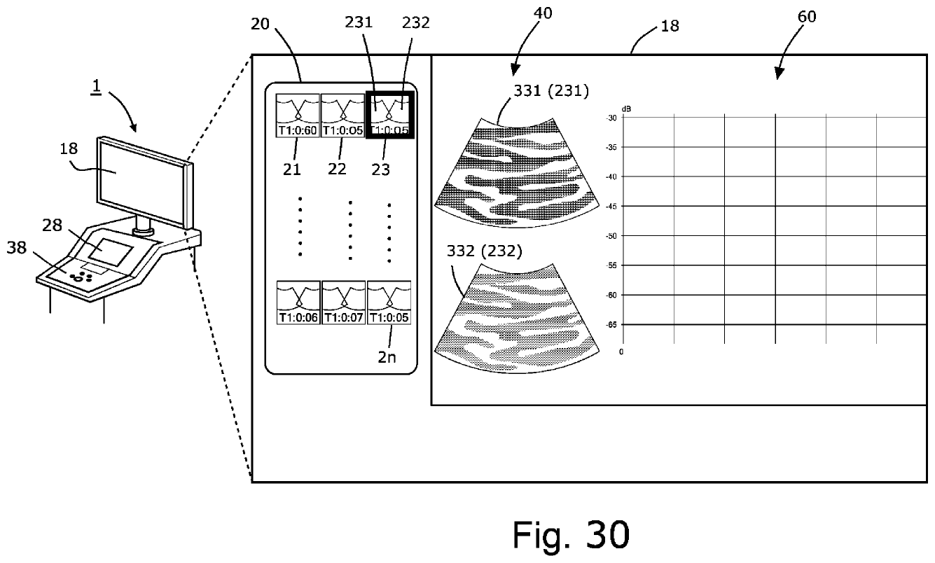

In step ST5, the processor 7 reads the selected image group 23 and displays an analysis screen for analyzing the time-intensity curve of the selected image group 23 on the monitor 18 (see FIG. 30).

FIG. 30 is a diagram illustrating an analysis screen for analyzing the time-intensity curve of the selected image group 23.

Image group 23 includes an image group 231 of contrast images and an image group 232 of B-mode images, with contrast image 331 from image group 231 displayed in the upper part of the image display region 40 and B-mode image 332 from image group 232 displayed in the lower part. Image groups 231 and 232 are reproduced as video with time length TS₃ (see FIG. 4). In the image display region 40, a single contrast image 331 (an image of one frame selected from a plurality of frames) from image group 231 may be displayed as a still image, while a single B-mode image 332 from image group 232 may be displayed as a still image. Alternatively, only one image group 231 of contrast images and image group 232 of B-mode images may be displayed.

After displaying contrast image 331 and B-mode image 332 from image group 23, the process proceeds to step ST6.

In step ST6, the user performs analysis work to analyze the time-intensity curve in the selected image group 23. The analysis operation is performed in the same manner as that described above, so further description has been omitted. Upon completion of the analysis work, the process proceeds to step ST7, where the user determines whether or not to select another image group. When it is decided that an image is to be selected, the process returns to step ST2.

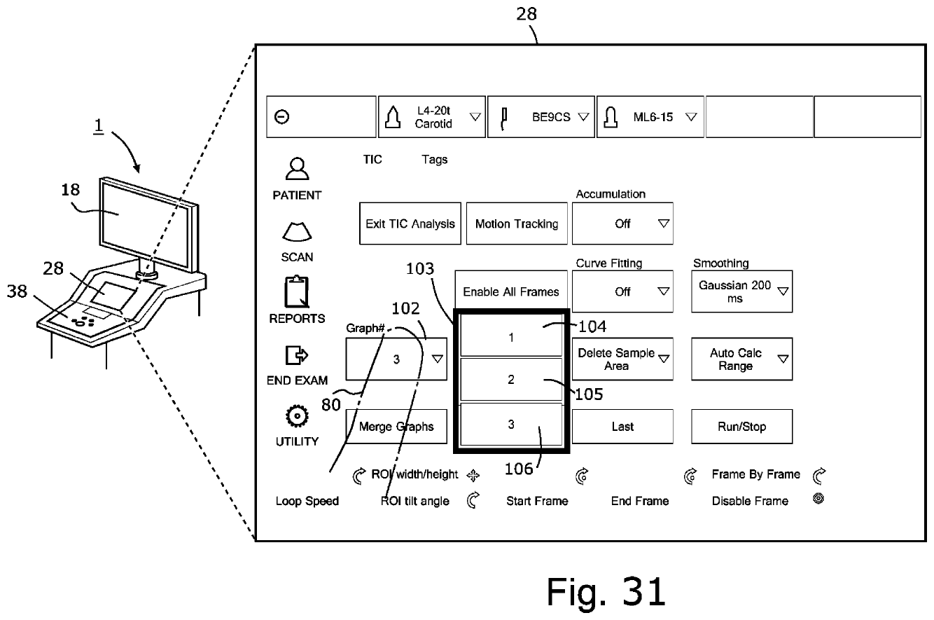

Returning to step ST2, when the user wants to modify a previously selected image group, for example, when the user wants to select image group 22, the user touches button 102 on the touch panel 28 with a finger 80 as illustrated in FIG. 31. When the user touches button 102, a sub-menu 103 is displayed to allow the user to reselect an image group previously selected by the user. In the sub-menu 103, three selection buttons 104, 105, and 106 are displayed. Selection buttons 104 and 105 are buttons for selecting image groups 21 and 22, respectively, as described with reference to FIG. 25. Meanwhile, selection button 106 includes image number 3. Image number 3 indicates that selection button 106 is a button that allows the user to reselect image group 23 (see FIG. 29) to which image number 3 has been assigned. When the user wants to correct previously selected image group 22, the user touches selection button 105. When selection button 105 is touched, a correction screen for image group 22 is displayed on the monitor 18. In this way, the user can perform correction work on the data set for image group 22.

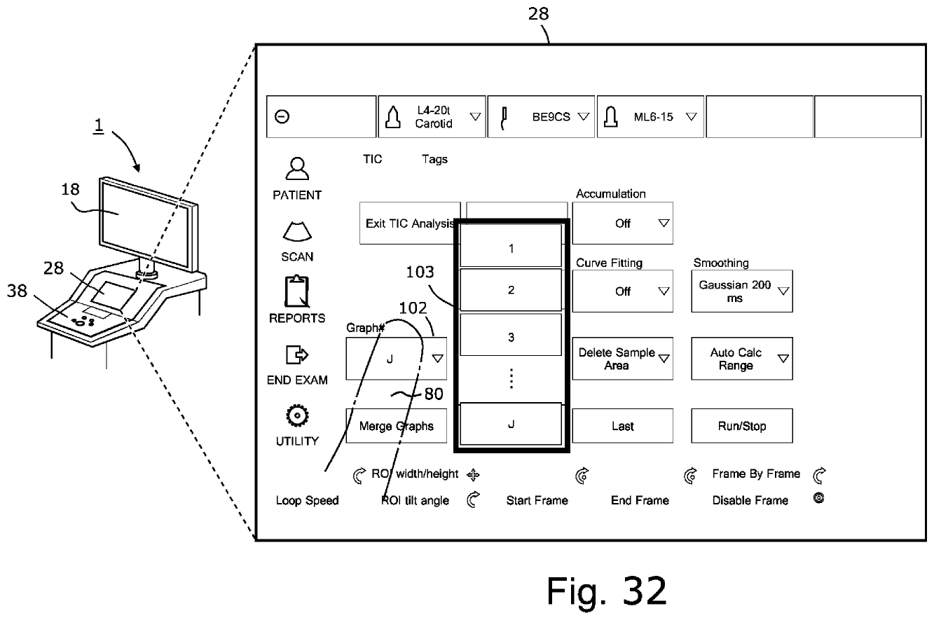

In a similar manner, steps ST2 to ST10 are repeated until further selection of image groups is deemed unnecessary by the user in step ST7. While steps ST2 to ST10 are being repeated, if the user wants to execute correction work on a previously selected image group, as illustrated in FIG. 32, the user touches button 102 on the touch panel 28 with a finger 80. When the user touches button 102, a sub-menu 103 is displayed to allow the user to reselect an image group previously selected by the user. In the sub-menu 103, J selection buttons are displayed. In this way, the user can cause the monitor 18 to display a correction screen for a previously selected image group by touching the button corresponding to the image group to be selected from among the J selection buttons.

In step ST7, if the user determines that it is not necessary to select an image group, the process proceeds to step ST11. For example, in the event the user has selected all the images necessary for the diagnosis of a patient and thus determines that subsequent images do not have to be selected, the process proceeds to step ST11.

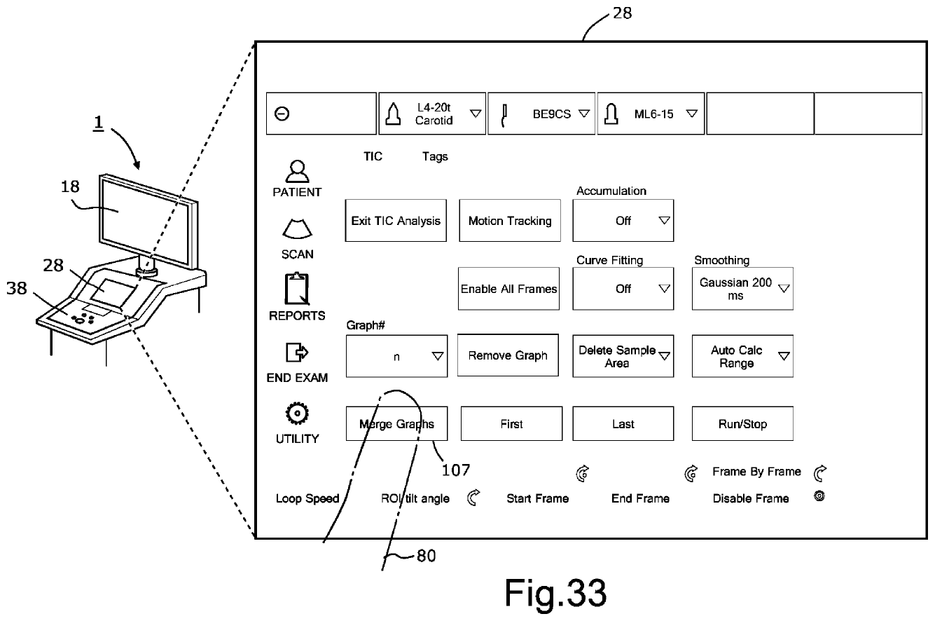

In step ST11, the user operates the user interface to input a command to display a plurality of data sets obtained by executing steps ST1 to ST10 as a single integrated data series. In the present embodiment, for example, as illustrated in FIG. 33, the user can input a command to display a plurality of data sets as a single integrated data series by touching the Merge Graphs button 107 displayed on the touch panel 28. When the user interface receives this command, the processor arranges the plurality of data sets in a time series, integrates them, and displays them as a single integrated data series.

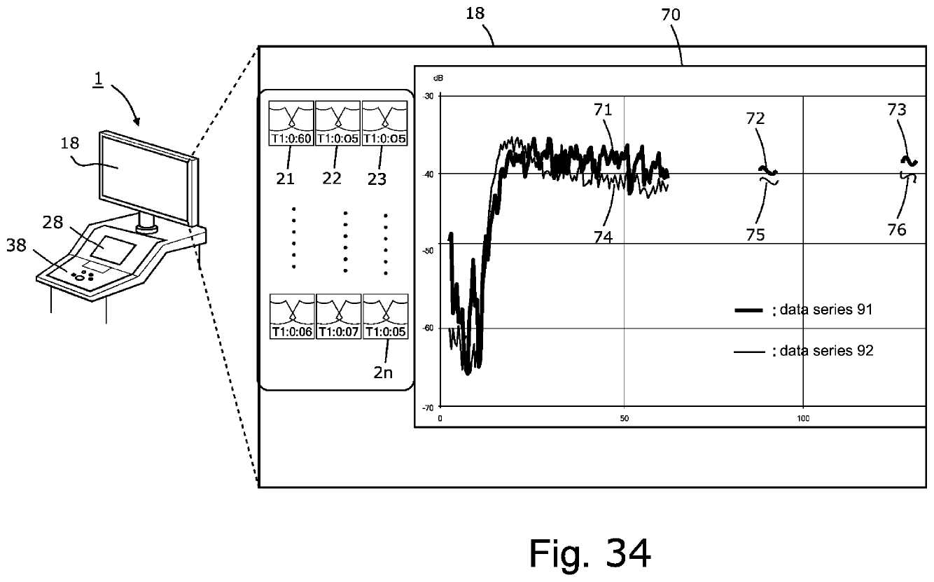

FIG. 34 is a diagram illustrating a plurality of data sets displayed as a single integrated data series.

A merge graph region 70 is displayed on the monitor 18. Data series 91 and 92 are displayed in the merge graph region 70. In FIG. 34, for convenience of explanation, data series 91 and 92 of data sets obtained from three image groups 21, 22, and 23 are illustrated.

The data series 91 is indicated by a thick line, while the data series 92 is indicated by a thin line.

The data series 91 represents the temporal change in a feature in a region of interest assigned to a target (for example, a tumor) in image groups 21, 22, and 23. The data series 91 includes a data set (time-intensity curve 71) obtained based on image group 21, a data set (time-intensity curve 72) obtained based on image group 22, and a data set (time-intensity curve 73) obtained based on image group 23.

Meanwhile, data series 92 represents the temporal change in a feature in a region of interest assigned to the tissue to be compared with the target. Data series 92 includes a data set (time-intensity curve 74) obtained based on image group 21, a data set (time-intensity curve 75) obtained based on image group 22, and a data set (time-intensity curve 76) obtained based on image group 23.

By referring to data series 91 and 92, the user can visually and easily recognize how a feature in a region of interest changes over time not only in the data set for each image group but also in a plurality of image groups 21, 22, and 23. This makes it possible to provide the user with more meaningful information for diagnosing a patient.

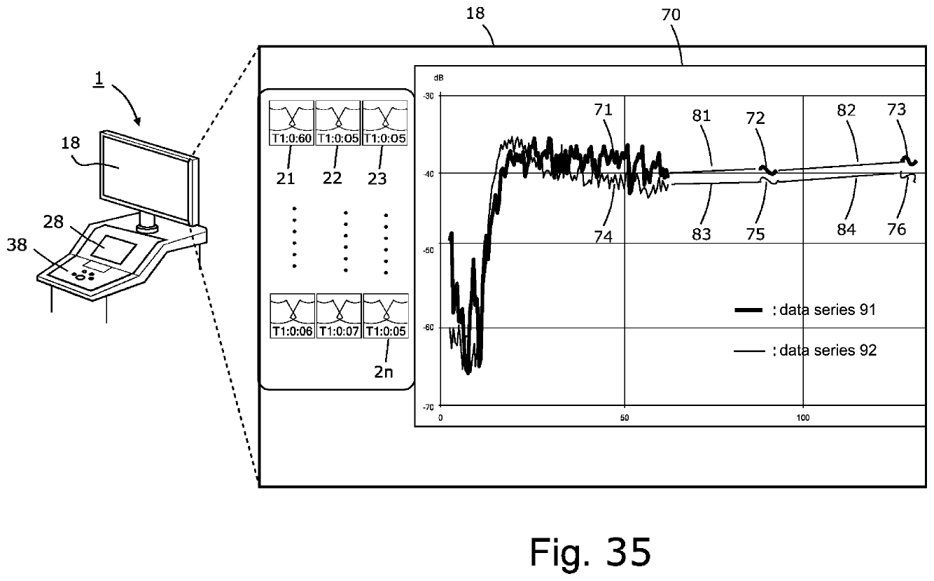

In FIG. 34, time-intensity curves 71, 72, and 73 of data series 91 are discretely displayed. Time-intensity curves 74, 75, and 76 of data series 92 are also discretely displayed. However, time-intensity curves adjacent to each other in the time axis direction may be connected by a line. The user may operate the user interface to input a command for adding a line connecting time-intensity curves. FIG. 35 is a diagram illustrating lines 81, 82, 83, and 84 added to connecting time-intensity curves. By adding these lines, each of data series 91 and 92 can be displayed as a single integrated time-intensity curve. In addition to or instead of a time-intensity curve, a differential curve (a first order differential curve, a second order differential curve, etc.) of a time-intensity curve can be displayed.

In this way, the flow in FIG. 4 ends.

In the present embodiment, when the user wants to modify the data set of a previously selected image group after having selected another image group, the user can display a correction screen for the previously selected image group simply by touching the button for the image group to be reselected on the touch panel 28. In this way, the user can easily correct the data set of a previously selected image group.

In the present embodiment, the user reselects a previously selected image group by touching a selection button (for example, selection button 104) displayed on the touch panel 28. However, the selection button may be displayed on the display 18 instead of the touch panel 28 and the operation panel 38 may be configured so as to receive input from the user to reselect the image group corresponding to the image number of a selection button displayed on the display 18.

In the present embodiment, the user selects a new image group by operating the operation panel 38. However, a new image group may be selected by operating the touch panel 28.

In the present embodiment, when the image group selected by the user is a newly selected image group in step ST3, the process proceeds to step ST4 and an image number is assigned to the image group selected by the user. However, as long as the newly selected image group can be identified, an identifier different from an image number may be assigned to an image group selected by the user. For example, the elapsed time from the start of administration of a contrast agent to the start of data acquisition may be assigned as an identifier to an image group selected by the user. When elapsed time is used as the identifier, a selection button including elapsed time instead of an image number can be displayed on the touch panel 28. In this case, the touch panel 28 may be configured such that, when the user touches a button displayed on the touch panel 28, the touch panel 28 receives input from the user to reselect the image group corresponding to the elapsed time. Alternatively, a selection button including elapsed time may be displayed on the display 18 instead of the touch panel 28, so when the user operates the operation panel 38 to input a command to select a selection button, the operation panel 38 may receive input from the user to reselect an image group corresponding to the elapsed time on a selection button displayed on the display 18. By displaying selection buttons including the elapsed time as the identifier, the user can visually and easily understand to which image group of data acquisitions SC1 to SCn (see FIGS. 3 and 4) the selection button displayed on the touch panel 28 or the display 18 corresponds.

### REFERENCE SIGNS LIST

1 Ultrasound diagnostic device
2 Ultrasound probe
2a Vibrating element
3 Transmission beamformer
4 Transmitter
5 Receiver
6 Reception beamformer
7 Processor
8 Display
9 Memory
10 User interface
15 External storing device
18 Display monitor
20 Thumbnail region
21, 22, 23, 2n Image group
28 Touch panel
30, 40 Image display region
38 Operation panel
41, 42, 43, 44, 51, 52, 53, 54 Region of interest
56 Operator
57 Subject
60 TIC region
61 Data set
62, 63, 64, 66, 67, 68, 69, 71, 72, 73, 74, 75, 76, 661 Time intensity curve
70 Merge graph region
80 Finger
81, 82, 83, 84 Line
91, 92 Data series
101, 102, 107 Button
103 Sub-menu
104, 105, 106 Selection button
211, 212, 221, 222, 231, 232 Image group
311, 321, 331 Contrast image
312, 322, 332 B-mode image

1. An ultrasonic diagnostic device comprising: a user interface;
a processor;
a storage device configured to store a plurality of image groups, each image group including a plurality of ultrasound images arranged in a time series; and
a display,
wherein the processor is configured to:
(i) accept, through the user interface, an input from a user selecting one image group from among the plurality of image groups,
(ii) responsive to the input selecting an image group, display the ultrasound images included in the selected image group on the display,
(iii) accept, through the user interface, an input for assigning a region of interest to an ultrasound image displayed on the display,
(iv) responsive to the input assigning the region of interest, create a data set representing a temporal change in a feature in the region of interest based on the ultrasonic images included in the selected image group, and
(v) arrange and integrate a plurality of data sets obtained by repeatedly executing operations (i) to (iv) in a time series, then create a data series representing a temporal change in a feature in the region of interest in the plurality of data sets,
wherein the user input to select one image group includes a first input from the user to select a first image group of the plurality of image groups and a second input from the user to select a second image group of the plurality of image groups after accepting the first input, and
wherein the user interface is configured to accept user input in order to reselect the first image group after the second input has been accepted, so that the user can perform correction work on the data set representing the temporal change in the feature in the first image group selected by the first input that was accepted before the second input.

2. The ultrasonic diagnostic device according to claim 1, wherein the processor is further configured to determine whether the one image group selected by the user is a newly selected image group, and if not a newly selected image group, determines that it has accepted input to reselect the first image group and reads and restores the analysis information mapped to the first image group from the storage device.

3. The ultrasonic diagnostic device according to claim 2, wherein when an image group selected by the user among the plurality of image groups is a newly selected image group, the processor is further configured to assign an identifier to the image group selected by the user.

4. The ultrasonic diagnostic device according to claim 3, wherein the identifier comprises
an image number, or
the elapsed time from the start of administration of a contrast agent to the start of data acquisition.

5. The ultrasonic diagnostic device according to claim 4, wherein when an image group selected by the user among the plurality of image groups is a newly selected image group, the processor is further configured to interconnect a parameter indicating the image number of the newly selected image group.

6. The ultrasonic diagnostic device according to claim 4, wherein the user interface comprises a touch panel,
the touch panel is configured to display a button for reselecting a selected image group, the button including the image number, and
when the user touches the button, the touch panel configured to accept input from the user to reselect the image group corresponding to the image number.

7. The ultrasonic diagnostic device according to claim 4, wherein the user interface comprises an operation panel,
a button for reselecting a selected image group is displayed on the display, the button including the image number, and
when the user operates the operation panel and inputs a command to select the button, the operation panel accepts the user input to reselect the image group corresponding to the image number.

8. The ultrasonic diagnostic device according to claim 4, wherein the user interface comprises a touch panel,
the touch panel is configured to display a button for reselecting a selected image group, the button including the elapsed time, and
when the user touches the button, the touch panel is configured to accept input from the user to reselect the image group corresponding to the elapsed time.

9. The ultrasonic diagnostic device according to claim 4, wherein the user interface comprises an operation panel,
a button for reselecting a selected image group is displayed on the display, the button including the elapsed time, and
when a user operates the operation panel to input a command to select the button, the operation panel accepts input from the user to reselect the image group corresponding to the elapsed time.

10. The ultrasonic diagnostic device according to claim 3, wherein the user interface accepts input from the user to newly select an image group.

11. The ultrasonic diagnostic device according to claim 1, wherein the display displays a thumbnail corresponding to each of the plurality of image groups.

12. The ultrasonic diagnostic device according to claim 2, wherein analysis information for analyzing the first image group is mapped to the first image group and stored in the storage device, and
when the user interface accepts input from the user to reselect the first image group, the processor reads and restores the analysis information mapped to the first image group from the storage device.

13. The ultrasonic diagnostic device according to claim 12, wherein the analysis information includes at least one of the position, size, shape, or color of a region of interest, a degree of smoothing for a time-intensity curve, and whether motion compensation processing has been performed on the region of interest.

14. The ultrasonic diagnostic device according to claim 1, wherein the display displays a time-intensity curve obtained by connecting data adjacent to each other in the time axis direction in the data included in the data set with a line.

15. The ultrasonic diagnostic device according to claim 12, wherein the processor updates the corrected analysis information when correction work has been performed by the user operating the user interface, and
the correction work includes at least one of assigning a new region of interest, changing the position, size, shape, or color of a region of interest, changing the degree of smoothing for a time-intensity curve, and changing whether motion compensation processing has been performed on a region of interest.

16. A method for operating an ultrasonic diagnostic device including a user interface, a processor, a storage device that stores a plurality of image groups, each image group including a plurality of ultrasound images arranged in a time series, and a display, the method comprising:
(i) obtaining an input from a user selecting one image group from among the plurality of image groups;
(ii) responsive to the user interface has accepted input from the user selecting the one image group, displaying the ultrasound images included in the selected image group on the display;
(iii) receiving an input from a user performing an operation of assigning a region of interest to the ultrasound image displayed on the display;
(iv) responsive to the user interface accepting input from the user assigning the region of interest, creating a data set representing a temporal change in a feature in the region of interest based on the ultrasonic images included in the selected image group, and
(v) integrating a plurality of data sets obtained by repeatedly executing (i) to (iv) in time series, then creating a data series representing a temporal change in the feature in the region of interest in the plurality of data sets,
the user input to select one image group including a first input from the user to select a first image group from the plurality of image groups and a second input from the user to select a second image group from the plurality of image groups after accepting the first input,
and the method further comprising:
accepting, after the user interface has accepted the second input, input from the user to reselect the first image group so that the user can perform correction work on the data set representing the temporal change of the feature in the first image group selected in the first input before the second input was accepted.

17. A non-transitory computer readable medium configured to be executed by a computer to:
(i) obtain an input from a user through a user interface selecting one image group from among the plurality of image groups;
(ii) responsive to the user interface accepting input from the user selecting the one image group, display the ultrasound images included in the selected image group on the display;
(iii) receiving an input from a user performing an operation of assigning a region of interest to the ultrasound image displayed on the display;
(iv) responsive to the user interface accepting input from the user assigning the region of interest, create a data set representing a temporal change in a feature in the region of interest based on the ultrasonic images included in the selected image group, and
(v) integrate a plurality of data sets obtained by repeatedly executing (i) to (iv) in time series, then create a data series representing a temporal change in the feature in the region of interest in the plurality of data sets,
the user input to select one image group including a first input from the user to select a first image group from the plurality of image groups and a second input from the user to select a second image group from the plurality of image groups after accepting the first input,
and the non-transitory computer readable medium may be further configured to be executed by a computer to: accept, after the user interface has accepted the second input, input from the user to reselect the first image group so that the user can perform correction work on the data set representing the temporal change of the feature in the first image group selected in the first input before the second input was accepted.
